(19) United States
(12) Patent Application Publication
(10) Pub. No.: US 2025/0262566 A1
(43) Pub. Date: Aug. 21, 2025

(54) DEVICE FOR SEPARATING AN ANALYTE FROM OTHER COMPONENTS IN AN ELECTROLYTIC SOLUTION

(71) Applicant: Nyctea Technologies AB, Göteborg (SE)

(72) Inventors: Gustav Ferrand-Drake Del Castillo, Göteborg (SE); Maria Kiriakidou, Mölndal (SE); Andreas Dahlin, Askim (SE)

(21) Appl. No.: 18/702,958

(22) PCT Filed: Oct. 21, 2022
(86) PCT No.: PCT/SE2022/050955

§ 371 Date: Apr. 19, 2024

(30) Foreign Application Priority Data
Oct. 21, 2021 (SE) 2151286-8

(51) Int. Cl. B01D 15/38; B01D 15/16; B01D 15/42; G01N 27/30; G01N 27/333

(52) U.S. Cl. CPC B01D 15/3885; B01D 15/168; B01D 15/424; G01N 27/302; G01N 27/333; B01D 15/3804

(57) ABSTRACT

A device (100, 100′, 100″) for separating an analyte (200) from other components in an electrolytic solution. The device comprises a housing (114, 115, 116, 117, 118, 119) provided with a solution inlet (104) and a solution outlet (105); a working electrode (101) arranged in the housing such that an electrolytic solution arranged to flow (F) from the inlet to the outlet contacts at least a portion of the working electrode; a counter electrode (102) arranged in the housing (114, 115, 116, 117, 118, 119). At least a portion of a surface of the working electrode (101) is provided with a polyelectrolytic coating (111), the polyelectrolytic coating (111) being arranged to upon application of a potential difference between the working electrode (101) and the counter electrode (102) switch between a first and second state, wherein in the first state an analyte (200) is captured in the polyelectrolytic coating (111) and in the second state a captured analyte (200) is released from the polyelectrolytic coating (111).

### TECHNICAL FIELD

The present document relates to a device for non-invasive separation and concentration of an analyte, e.g. a protein pharmaceutical, a viral gene vector, extracellular vesicle carbohydrate or oligonucleotide, in an electrolytic solution, a system comprising such a device and a method using such device.

### BACKGROUND

An urgent global challenge is to supply an unmet and rapidly increasing demand for new biological pharmaceuticals e.g. antibodies, gene vectors and vaccines. Biological pharmaceuticals are currently expensive and slow to produce, which greatly limits global and widespread use of these life-saving therapies. A highly cost-intensive and rate-limiting step of the production is the downstream purification by multiple steps of chromatography after harvest of the cell culture.

Chromatography is a separation method involving a solid support material and a mobile fluid phase composed of a mixture of different chemical species to be separated. Many different physical and/or chemical interactions between the solid support and the molecules of the mixture will govern the rate of migration of the various components of the fluid through the solid support material. This allows for many different modes of chromatography that uses different mechanisms to achieve separation. In general, the surface chemistry of the solid support is prepared such that one specific target molecule, or one type of molecule with a certain chemical feature, adheres strongly by intermolecular attractions e.g. electrostatic attraction, ligand interactions, to the surface of the solid support while the remaining components of the mixture solution are flushed through the chromatography column resulting in a successful separation. However, to retrieve the pure sample of the target molecule elution of the bound material from the chromatography solid support surface is required. This is typically achieved by injection of some chemical that disrupts the interaction between the target molecule and the solid support allowing collection of the target molecule from the outflow of the chromatography column.

Chromatography is extensively used to separate biological pharmaceuticals, common types include affinity, ion-exchange, size exclusion, reversed phase and hydrophobic interaction chromatography. Monoclonal antibodies constitute the largest class of protein pharmaceuticals and in its downstream purification, protein A affinity chromatography is standard followed by a sequence of different polishing chromatography steps (e.g. ion exchange, size exclusion).

Affinity chromatography, especially those that incorporate protein ligands are extremely expensive due to the high cost of the chromatography resin (solid support). Whenever possible to reduce production costs, a cheaper combination of chromatography steps that avoids affinity chromatography with satisfactory purity is preferable. However, due to extremely high demands on purity on the end-product from impurities like host cell proteins, endotoxins, and serum contaminants, affinity chromatography is almost always a requirement since it offers extremely high specificity in a single step (Kelley, B. (2007) ‘Very large scale monoclonal antibody purification: The case for conventional unit operations’, Biotechnology Progress, 23 (5), pp. 995-1008). For instance, protein A chromatography remains the standard chromatography method for monoclonal antibodies, despite being the largest over-all cost contributor to the separation process.

A short-coming of most chromatography methods of biomolecules is the lack of efficient methods for eluting, meaning removal of the target biomolecule from the solid support surface without damaging or contaminating the product in the process.

Biomolecules are particularly difficult to elute since many targets have a highly sensitive secondary and tertiary structure where retention of this structure is critical for their proper functionality in terms of therapeutic effect or catalytical function. Currently there are no affinity chromatography methods that does not use elution by some chemical additive. For recombinant polyhistidine-tagged proteins immobilized metal-ion affinity chromatography is a standard chromatography step where elution is achieved by injection of an imidazole 250-1000 mM solution to disrupt the resin-protein bond. For protein A chromatography, pH 2-3 buffer is flushed through the chromatography column to elute antibodies. Even for other types of chromatography like ion-exchange and hydrophobic interaction chromatography it is common to perform elution at very high salt concentrations (500-1000 mM) of monovalent salts e.g. KCl, NaCl or surfactants. Addition of chemicals to produce elution are added to the product in the outlet feed of the chromatography column. In many cases long exposure to very low pH or reactive chemicals like imidazole pose a risk for aggregation contamination and reduced yield of the pharmaceutical product.

Chemical additives are particularly challenging in the manufacturing of biological pharmaceuticals where extremely high demands are placed on purity to comply with patient safety and regulatory demands. For instance, in metal ion chelation chromatography there is a substantial risk that the metal ions used to bind poly-histidine tagged proteins (e.g. Ni²⁺ and Cu²⁺) leaches into the eluent and causing downstream contamination of the products. Additional post-processing polishing operations to remove chemical additives, prevent potential damage and retrieve a pure product adds significant process time. Therefore, new protocols for purification that reduces the need of additives and thereby of post-processing steps have high potential to improve the economy of production. Affinity chromatography of other proteins than antibodies require recombinant engineering to introduce the necessary tags for their purification (e.g. glutathione, streptavidin tag systems). Although several methods have been developed to attach fusion proteins and peptide tags, non-natural amino acid sequences require removal and proof of their removal in order to comply with good manufacturing practices. Thus, it is preferable whenever possible to use a separation that achieves high purity and yield without requiring fusion tags and the resource demanding protocols associated with their removal.

Despite the shortcomings of chromatography for purification of proteins, it is used for industrial purification of proteins where acceptable yield can be achieved, despite the use of harsh chemicals. A high demand compensates for inefficiencies in the manufacturing process. Modern biopharmaceuticals have, however, become increasingly complex and place higher demands on the production and purification process. One notable example is viral vectors, such as Adeno-associated viruses (AAVs), for gene therapy and cancer treatment. Available solutions do not fit the desired needs for purity, speed and cost of production. Elution with chemicals is the main obstacle for using traditional chromatography to purify large biomolecular assemblies like viral vectors, extracellular vesicles and cells. Viruses are three-dimensional assemblies of hundreds of proteins that encapsulate genetic material, which in turn may be enveloped by a lipid membrane. The virus structure therefore has more narrow conditions for stability compared to single proteins. Viral vectors may not tolerate high salt concentrations, surfactants, and pH gradients. Another major problem is the larger size of biomolecular assemblies. Viral vectors are 10 to 100 times larger than protein pharmaceuticals, challenging the structure of chromatography beads and resins. The conventional chromatography resins have too tiny pores for viruses, introducing diffusion limitations with long process times as a result and a substantial risk of clogging the chromatography column.

For all chromatography applications a major issue is low concentration of the eluted product and a need for removal of the elution chemical. To up-concentrate diluted samples, centrifugation and filtration are used. Dialysis can be used for buffer exchange. Each of these additional process steps are, however, associated with additional yield losses, adding to the over-all cost of goods.

Chromatography separation applied on large scale production is extremely inefficient with respect to process-mass-intensity. A recent study by leading biopharmaceuticals companies found that production of 1 kg of a commercial monoclonal antibody required nearly 10 000 kg of input material, not including cleaning operations. More than >60% originates from the downstream purification process where water consumption is the greatest contributor followed by chemicals and consumables. Pharmaceuticals under development can be expected to have much larger process mass intensities. Bind-and-elute by use of chemicals is the main reason for the large consumption of water and chemicals where frequent washing, equilibration, and conditioning steps are needed to complete the purification cycle.

Electrochemical signals may be used to release biomolecules from electrodes (Bellare, M. et al. (2019) ‘Electrochemically stimulated molecule release associated with interfacial pH changes’, Chemical Communications. Royal Society of Chemistry, 55(54), pp. 7856-7859.). Reductive potentials may be used to increase the pH at the interface of the electrode, produce a basic pH gradient. Oxidative potentials may be used to reduce the pH at the interface of the electrode, produce an acidic pH gradient. The pH gradient established will depend on the composition of the solution. Importantly, the concentration of buffer species will affect the extension of the pH gradient, and the concentration of redox active species and its mass transport to the surface of the electrode.

Such known devices are limited to low loading of molecules and prevent binding of large capacities necessary for practical applications. Furthermore, the release is single-use where the electrode lacks the capacity for repeated use. A major limitation of the electrochemical release surfaces of protein biomolecules is the lack of surface coatings that can reliably sustain the structure of the biomolecule on the surface without unfolding and degradation due to strong hydrophobic interactions with the surface during binding.

It is widely acknowledged that there is a need for new separation techniques of biomolecules that can complement or replace cost-intensive and time consuming parts of biotherapeutics production, in particular chromatography. A new separation technique should preferably achieve high yield and specificity, be fast, reduce or eliminate the need of chemicals of environmental and health concern, and reduce or eliminate additional post-processing steps such as up-concentration and buffer exchange. The technology should be scalable, meaning it is possible to go from small scale, μg-mg, to large scale, g-kg, without escalating the costs of goods or the environmental burden and use of input materials.

### SUMMARY

It is an object of the present disclosure to provide an improved or at least an alternative device, system and method for preparative scale separation and concentration of analytes, such as biomolecules, from other components in an electrolytic solution.

The invention is defined by the appended independent patent claims. Non-limiting embodiments emerge from the dependent claims, the appended drawings and the following description.

According to the first aspect there is provided a device for separating an analyte from other components in an electrolytic solution. The device comprises a housing provided with a solution inlet and a solution outlet; a working electrode arranged in the housing in a space between the solution inlet and the solution outlet, and arranged such that an electrolytic solution arranged to flow from the inlet to the outlet contacts at least a portion of the working electrode. A counter electrode is arranged in the housing in a space between the inlet and the outlet at a distance from the working electrode, and arranged such that it is in electrical connection with the working electrode via the electrolytic solution arranged to flow from the inlet to the outlet. At least a portion of a surface of the working electrode is provided with a polyelectrolytic coating, the polyelectrolytic coating being arranged to upon application of a potential difference between the working electrode and the counter electrode switch between a first and second state, wherein in the first state an analyte is captured in the polyelectrolytic coating and in the second state a captured analyte is released from the polyelectrolytic coating.

The analyte may for example be a biological substance. The analyte may be an oligonucleotide, a protein, a gene vector, a lipid nanoparticle, liposome, a carbohydrate, a glycosylated biomolecule, or hydrogen bonding macromolecule of synthetic or biological origin provided in an electrolytic solution comprising other components such as other biomolecules and/or chemicals. The protein may be a protein pharmaceutical. The protein may be a protein that has an implicated important role in the understanding of a disease, for understanding characterizing or developing treatments against a certain disease, such as but not limited to Parkinson's or Alzheimer's disease. The lipid nanoparticle may be a drug delivery carrier. The liposome may be a liposome drug delivery carrier. The analyte may be a fusion or conjugate product between a synthetic molecule or polymer and a protein or an oligonucleotide.

If the analyte is a protein or a construction predominantly composed of proteins or lipids, like a virus particle or an exosome, the analyte is captured in the polyelectrolytic coating in the first, neutral state through non-electrostatic binding, e.g. hydrogen bonds, and in the second, charged state, the captured analyte is released from the polyelectrolytic coating by electrostatic repulsion. Conversely, if the analyte is a carbohydrate or oligonucleotide the analyte is captured to the polyelectrolytic coating in the first, charged state followed by release upon a switch to the second, neutral state. If the analyte is a fusion of two different kinds of biomolecules, for example an antibody conjugated to an oligonucleotide, either mode of capture and release is possible and which will be practical to use depends on which of part of the molecule dominates the interaction. Finally, if the polyelectrolytic coating is post-functionalized with a biological ligand molecule the physiochemical binding characteristic of the ligand-biomolecule pair as a function of pH will dictate the condition for capture and release.

Electrolytic solution is a solution that generally contains ions, atoms or molecules that have lost or gained electrons, and is electrically conductive. The electrolytic solution is preferably entirely free from chemicals that trigger elution of the analyte. Such electrolytic solution may for example be a cell culture medium, buffer solution etc. The electrolyte is composed of a buffering species, everything from 1 mM (extremely low), to physiological buffer concentrations of 100 mM and up to 1 M may work. Salt, ions, needs to be in the electrolyte as carrier of charges. The total salt concentration, the ionic strength, will influence the pKa of the polyelectrolytic coating. A high salt concentration leads to high pKa and a low salt concentration leads to low pKa, changing the pivot point between the first (neutral) stage and the second (charged state)—meaning the pH at which point the polyelectrolytic coating is analyte binding and repelling.

To enable electrochemical reactions, the electrolytic solution comprises redox-active species. Such redox-active species may either be inherently present in the electrolytic solution, e.g. oxygen, or glucose, or may be added to the electrolytic solution, e.g. hydroquinone, hydrogen peroxide, dopamine hydrochloride (DOPA), ascorbic acid, 4-aminophenethyl alcohol (tyrosol), 3,4-dihydroxyphenetylacetic acid (DOPAC), β-nicotinamide adenine dinucleotide, oxygen and reduced disodium salt hydrate (NADH).

A redox-active species must be present to be able to switch the polyelectrolytic coating from the charged, high surface pH state to the neutral, low surface pH state. To switch between the neutral and charged state, redox-active species are present in the solution in the form of oxygen.

The polyelectrolytic coating may be arranged on any surface or part of surface of the working electrode, on some or on all surfaces of the electrode. If the electrode is porous, the polyelectrolytic coating may also extend into the pores of the electrode.

The polyelectrolytic coating may be in the form of a polyelectrolyte brush, a film, a gel, or layer. A thickness of such polyelectrolytic coating may be any value between a very thin coating on the nanoscale (˜1 nm) up to micrometers thick (˜1 μm).

The polyelectrolytic coating is a stimuli-responsive coating, which is arranged to, upon application of a potential difference between the working electrode and the counter electrode, switch between a first and second state. For a protein analyte, in the first state, a neutral state, an analyte is captured in the polyelectrolytic coating through non-electrostatic binding and in the second state, a charged state, a captured analyte is released/eluted from the polyelectrolytic coating through electrostatic repulsion. For a carbohydrate-containing analyte, in the first state, a charged state, an analyte is captured in the polyelectrolytic coating and in the second state, a neutral state, a captured analyte is released/eluted from the polyelectrolytic coating.

As immobilization of for example a protein analyte in the polyelectrolytic coating is mediated by non-electrostatic bonds when the polyelectrolytic coating is in a first, neutral state, this enables binding of proteins in the polyelectrolytic coating in their native state.

With the device high amounts (multilayers) of proteins may be spontaneously immobilized in their native state by non-electrostatic intramolecular attractive interactions e.g. hydrogen bonding, to the polyelectrolytic coating when in its neutral, protonated state. The proteins are irreversibly bound in/to the polyelectrolytic coating granted that the polyelectrolytic coating stays in its neutral state with preserved structure and catalytic function. When changed to its second, charged state the analytes are repelled from the coating.

The device described may be used for scalable and repeatable separation of analytes in a wide range of quantities (μg to kg) by adjustment of the size of the electrodes (and the area coated with polyelectrolytic coating).

The working electrode is regenerated by repelling/releasing/eluting the captured analyte when switched into its second state, keeping the polyelectrolytic coating on the electrode surface. Thereby, the device can be used a repeated number of times using the same working electrode coated with the very same polyelectrolytic coating. No chemicals of environmental and health concern are needed for releasing/removing the captured analyte from the working electrode.

The analyte may be selected from a protein, a lipid particle, an oligonucleotide, a carbohydrate, or any combination thereof.

The polyelectrolytic coating arranged on the surface of the working electrode may comprise a pH-responsive polymer covalently bound to the surface of the electrode through a monolayer of aryl bonds.

The polyelectrolytic coating may be covalently bound to the working electrode. The polyelectrolytic coating may be linked covalently to an electrode surface by an electrochemically insensitive monolayer of aryl bonds, such as for instance the diazonium salt surface functionalization.

The electrochemically stable chemical anchor i.e. the electrochemically insensitive bond comprising an aryl, enables tunable release of the captured analytes. Due to these electrochemically stable aryl bonds, the device and the polyelectrolytic coating on the working electrode surface can be reused a large number of times.

The polyelectrolytic coating has previously been described in WO2021/107836.

Through the application of a potential difference between the working electrode and the counter electrode, a local microscale pH gradient is created that extends from the surface of the working electrode. The pH-sensitive/responsive polymer switches its state as a result of the local pH difference on the surface. The switch of the pH sensitive/responsive polymer results in either capture or release of said analyte from the surface of the electrode, which gives rise to a separation between said analyte and other components of the sample. The separation takes place due to a differing affinity towards the electrode for the analyte compared to other components in the sample solution. The difference in affinity comprises non-electrostatic intermolecular attractions, e.g. hydrogen bonding between the analyte and the polymer-coated electrode. Further, it may be due to electrostatic attraction or repulsion.

The pH-responsive polymer may be a polymer comprising a carboxylic acid group.

The pH-responsive polymers may be, e.g., polymers comprising carboxylic acid groups, which have ability to dissociate protons, or to uptake protons, as a result of the pH increasing or decreasing the at the interface of the electrode, respectively, the pH-responsive polymers being, for example, a poly(acrylic acid) (PAA) or a poly(methacrylic acid) (PMAA).

Immobilization of an analyte, such as a protein, in the polyelectrolytic coating is mediated by non-electrostatic, multivalent hydrogen bonds from carboxylic acid groups when the polyelectrolytic coating is in a first, neutral state, a protonated state, enabling proteins to bind in their native state.

The pH-responsive polymer may be a polymer functionalized with a pH-responsive and analyte-specific ligand.

The polymer, for example a polymer comprising carboxylic acid groups as discussed above, or other kinds of polymers e.g. poly(glycidyl methacrylate), poly(2-hydroxyethyl methacrylate), heparin, hyaluronic acid, dextran, can be modified/functionalized to contain functional groups that are pH-responsive and have affinity for the analyte of interest. In some cases, the polymer is functionalized with a molecule with several functional groups that creates a “handle” for gripping specific analytes. Such handles can be analyte-binding at one pH and be analyte-repellent in another state.

The side group of the monomer (repeat group of the polymer) may contain a functional group that can for example be used as a linker, such as carboxylic acids, epoxy groups, glycidyl functional groups, or 2-hydroxyethyl groups, to which linker an enzyme, nitrilotriacetic acid-metal ion²⁺ (NTA-Me²⁺), protein A, protein G, calmodulin, streptavidin etc. may be immobilized. Thereby, the polymer may be functionalized with an analyte-specific ligand being an enzyme, NTA-Me²⁺, protein A, protein G, calmodulin, or streptavidin.

Nitrilotriacetic acid-metal ion²⁺, NTA-Me²⁺, ligands interact strongly and specifically with His-tagged proteins in for instance a buffer composed of TRIS 50 mM and NaCl 250 mM. Elution/release of His-tagged proteins from a working electrode functionalized with the NTA-Me²⁺ may be achieved by application of a negative potential, resulting in a redox reaction of the coordination metal of the NTA-Me²⁺ ligand that disrupts the analyte-polymer bond resulting in elution of said analyte from the surface. Elution of the His-tagged proteins from the NTA-Me²⁺ surface may be accomplished by application of a positive potential in the presence of hydroquinone or a similar chemical that undergoes oxidation whereupon a local acidic pH gradient is produced, resulting in disruption of the pH sensitive metal ion chelation bond between the NTA-Me²⁺ and the His-tagged protein.

The polyelectrolytic coating may be functionalized with a biological molecule containing a ligand, such as but not limited to protein A, to achieve a highly specific interaction with target analyte, e.g. a monoclonal antibody, exosome, virus capsid, or an enzyme.

The pH-responsive and analyte-specific ligand may be an enzyme, NTA-Me²⁺, protein A, protein G, calmodulin, or streptavidin.

Using for example NTA-Me²⁺ or protein A functionalized polymers, the device can be used to separate or concentrate HIS-tagged proteins without using any harsh elution buffer. Only a potential difference is applied between the working electrode and the counter electrode to elute the analyte.

70-100% of the working electrode may overlap with the counter electrode, as seen in a plane orthogonal to a direction of flow of the electrolytic solution from the solution inlet towards the solution outlet.

Thereby a more efficient electrochemical reaction may be produced on the working electrode. By overlapping/intertwining of the working and counter electrode the distance of electrolytic solution may be minimized and the larger direct surface area exposure between the two electrodes may be accomplished.

The average distance between the electrodes may be 1 μm to 200 mm.

In one embodiment the average distance is a smaller distance of 1 μm to 20 μm, In another embodiment the distance may be 1 μm to 200 mm.

In the first case, only the working electrode produces a pH gradient when an electrochemical signal is applied. If the counter electrode does not give rise to an electrochemical pH gradient that counteracts/neutralizes the corresponding pH gradient produced on the working electrode, the distance between the electrodes may be separated by only a few micrometers. The distance may be 1-10 μm, 1-5 μm, 5-10 μm or 10-15 μm.

In the second case, the electrochemical signals produces opposite pH gradients on the working and the counter electrode.

When the counter electrode produces a pH gradient that counteracts the pH gradient on the working electrode upon applying an electrochemical potential the separation may need to be larger, on the scale of millimetres: the distance may be 1-10 mm, 10-20 mm, 20-200 mm. For instance the counter electrode may give rise to a basic pH gradient and the working electrode may give rise to an acidic pH gradient. If the liquid at the two interfaces are very close in contact and under a flow rate, the effect on the pH would be to neutralize, preventing release. If there is only one pH gradient produced by electrochemistry i.e. the counter electrode undergoes a faradaic reaction without proton generation/consumption or charge transfer occurs primarily by double layer charge build-up, there is no need to restrict the distance between the working and counter electrode.

An average thickness of the polyelectrolytic coating provided on the working electrode may be 10-50 nm.

An average thickness of the polyelectrolytic coating provided on the working electrode may be 10-50 nm, or 10-40 nm, or 10-30 nm, or 20-50 nm, or 20-40 nm, or 20-30 nm.

If the device is used for affinity chromatography applications, such a thickness of the polyelectrolytic coating enables release of the analyte during elution from the working electrode. A coating between 10-50 nm enables the pH gradient to reach a sufficiently high, or low pH value within the polyelectrolytic coating to disrupt the analyte-affinity ligand interaction.

The inner volume of the housing not occupied by the working electrode may be 5%-75%.

The inner volume of the housing not occupied by the working electrode may be 5-75%, or 10-75%, or 20-75%, or 30-75%, of 50-75%, or 5-70%, or 5-60%, or 5-50%, or 5-30%, or 5-20%, or 5-10%, or 10-30%.

This affects the possibility of concentrating the sample since the percentage of the inner volume of the housing that is not occupied by the working electrode contributes to a diluting effect of the analyte sample upon elution.

A lower percentage of working electrode within the inner volume of the housing may however be compensated or even over-compensated by the fact that the electrochemical potential instantaneously releases all of the bound analyte within a fraction of a second. The release may be as fast as microsecond by tuning of the electrochemical potential. The effect is a highly concentrated elution of analyte sample beyond what is possible by solution pH change which requires complete mixing of the interior to achieve release of analyte from the capture scaffold of the inner housing.

The working electrode may be porous and arranged in the housing such that the electrolytic solution is allowed to flow through the electrodes from the inlet through at least a portion of the working electrode to the outlet.

With a porous electrode the solution may be filtered through a micrometer aperture. Thereby, separation of large objects like impurities or aggregates could be achieved. A micrometer aperture also permits high surface area promoting high binding capacity of the analyte to the surface.

The working electrode may be solid but with a micro structured surface to boost binding capacity, whereby the flow passes in a tangential direction to the electrode surface.

A main direction of extension of the working electrode may extend in a direction substantially perpendicular to a flow direction from the solution inlet to the solution outlet.

The working electrode may have a porosity of 40% to 99%, and an electroactive surface area of the working electrode may be between 100 to 10,000 m²/m³.

The porosity may be 40%-99%, or 50-99%, or 60-99%, or 70-99%, or 80-99%, or 50-90%, or 50-80%, or 50-70%, or 50-60%, or 60-80%.

A high porosity, up to 99%, may be obtained by using for example a foam or sponge material for the electrode. A porosity of 40% or more may be obtained by using e.g. a mesh electrode.

The electroactive surface area of the working electrode may be between 100 to 10,000 m²/m³, or 500-10,000 m²/m³, or 1,000-10,000 m²/m³.

For higher porosities, improved mass transfer is achieved.

High porosity enhances convective mass transfer to the surface, reducing the need to rely on diffusion of the analyte to the surface, which is a slower process.

High porosity of the working electrode results in a reduced pressure-drop across the device, lowering the need of using high pressure fluidic components and reducing power consumption. Both pressure and effective mass-transfer are important for scaling up the device to g-kg scale.

The electroactive surface area may be substantially improved beyond the standard surface area of the working electrode if the porous internal surface area has a roughness on the nanoscale.

The working electrode preferably is highly porous, 95% while having a surface area of 5000 cm2/cm3 and where the pore structure is ordered.

The working electrode with a surface area of 100 to 10,000 m²/m³ has the ability to produce a pH gradient extending at least 5 micrometers away from the working electrode surface by electrocatalytical reaction with a reducing agent where the concentration of the reducing agent ranges from 1 nM to 100 mM.

Specific working electrode materials have different electrocatalytical abilities. It is preferable to select an electrolytic solution containing a reducing agent that matches the working electrode materials ability to electrocatalyse this compound to produce a pH gradient. For instance hydroquinone as reducing agent for producing acidic pH gradients has low electroactivity on stainless steel working electrodes, while it is highly electroactive on gold and platinum coated surfaces and nanoparticles. Ascorbic acid is efficiently electrooxidized on carbon-based electrodes to produce pH gradients while it is not useful to produce pH gradients that can efficiently trigger analyte elution on stainless steel, gold or platinum. Furthermore, some reducing agents work well on most substrates, oxygen is readily reduced on a wide variety of electrode materials to produce basic pH gradients.

If the working electrode is a porous foam structure it may have 10-100 pores per inch.

If the working electrode is a micrometer mesh structure the pore size may be between 10 and 0.01 micrometer.

The working electrode preferably has a pores or openings with size 1 micrometer. The pores or channels of the working electrode are preferably only open ended and no dead ends. The electrode may have pores which are ordered like manufactured in a weave pattern, or pores that are random, from a foaming type manufacturing process.

The counter electrode may be porous.

The working electrode and the counter electrode may be arranged in the housing such that the electrolytic solution arranged to flow from the inlet to the outlet first passes through the working electrode and then through or past the counter electrode.

The flow passes first through the working electrode, and then through or past the counter electrode. Thereby the flow direction of electrolytic solution does not counteract the desired pH gradient effect on the working electrode that leads to elution of analyte from the working electrode surface. The counter electrode may produce an electrocatalytical pH gradient that transports electrolytic solution which counteracts/neutralizes the electrocatalytical pH gradient established on the working electrode removing the desired elution effect of analyte from the working electrode surface.

The flow may pass substantially vertically through the working electrode structure.

A internal void space within the working electrode may be configured such that electrolytic solution passing through the working electrode creates an electrochemical pH gradient that is at least 1-20 μm large.

With internal void space of the working electrode is here meant the volume within the working electrode that can be occupied by electrolytic solution. The electrochemical pH gradient being 1-20 μm, or 1-10 μm or 1-5 μm or 5-10 μm.

The working electrode may have microporous structures, but it can also be stacked in a manner that reduces the chance to produce pH gradients between the sheets comprising the working electrode. It is therefore important that the stacking permits electrical connectivity throughout the entire electrode material, while also maintaining a sufficient void space that permits electrolytic solution to occupy a space of 1-20 um on the maximum surface area of the working electrode.

The working and counter electrode may be manufactured and incorporated into the device in a way that minimizes the void/dead volume in the housing, i.e. minimizes the internal volume of the housing that does not capture any analytes. By minimizing the void/dead volume in the housing, the electrochemical properties of the device are simultaneously optimized, promoting conditions by which electrochemical release with high concentration of the analyte can be achieved.

The device contains a counter electrode that may be of a porous material that is capable of high current/charge transfer capacity.

The counter electrode may have high electrostatic charge storage, i.e. storing charges in a double layer by being produced with a very large surface area and by suitable a material, like for instance activated carbon.

The counter electrode surface may have very high catalytical ability to transfer charge by faradaic electrochemical reactions for instance by being doped with highly catalytical elements like platinum.

The counter electrode may possess a combination of high electrostatic charge storage, and highly efficient faradaic charge transfer.

The counter electrode may conduct charge transfer by faradaic reactions that do not result in pH change.

The process solution may contain chemicals that enable electrocatalytical conversions on the counter electrode, which do not promote pH change on the counter electrode and therefore limits risk of interference with pH changes on the working electrode.

The counter electrode may be of a different material than the working electrode, where the counter electrode is optimized to have sufficient current capacity.

The device may contain a working and counter electrode made of the same material, in which case it is preferable if the effective surface area of the counter electrode is at least two times larger than the effective surface area of the working electrode to permit sufficient current capacity of the counter electrode to enable supply of current that allows setting a controlled specified voltage on the working electrode within the entire aqueous potential range±1.5 V.

The working electrode and the counter electrode are preferably inert materials that do not undergo permanent chemical changes during exposure to process solution, or during application of electrochemical signals.

The device may comprise a working electrode and counter electrode with different electrocatalytic abilities, permitting any volume relationship between the electrodes while still enabling a current density for setting specified voltages required for release of analytes.

The device may comprise engineered counter electrode with catalytic nanoparticles, or some other surface modification, that permit miniaturisation of the counter electrode with respect to the volume of the working electrode.

The device interior shell may be 3D printed in conductive material that permits integration of one or several electrode into the structure of the device walls, leading to further optimization of the device interior volume.

The device may be designed to have a sufficient volume contact between all of the electrodes in the system to permit efficient electrochemical properties of the device, while reducing the internal void volume increasing concentration of eluted analyte sample.

The device may be a three electrode set-up with a working electrode counter electrode and a reference electrode.

The device may have only two electrodes, a working electrode and a counter electrode, the reference electrode is omitted.

The device may further comprise a reference electrode arranged in the housing and arranged for electrical connection through the electrolyte solution with the working electrode and the counter electrode.

The reference electrode may have a stable and well-known electrode potential and it is used as a point of reference for the potential control and measurement. The working and counter electrodes may be arranged in the same electrolytic solution, and the reference electrode may be arranged in a separate tube containing a reference solution. The reference electrode may be made of a silver wire with a silver chloride coating (AgCl) or an electrode e.g. a carbon electrode coated with silver particles with an AgCl coating where the reference electrode is either directly exposed to the analyte solution, or it is separated by a semipermeable membrane through which ions can be transported but not the analyte or other molecules present in the analyte solution. For a reference electrode shielded by a semipermeable membrane the reference electrode solution used was 3M potassium chloride (KCl), which also it the solution used for storing the AgCl reference electrode. The reference electrode can in principle be any electrode with a stable and well-known reference electrode potential e.g. standard hydrogen electrode, saturated calomel electrode or a copper sulphate electrode. The working electrode, counter electrode and reference electrode preferably are manufactured and are composed of materials such that they do not actively or passively leach elements and compounds that are harmful for the downstream process, e.g. metal ions, radicals, and other compounds that may react with the analyte.

The reference electrode may be arranged at an average distance of 1-50 mm from the counter electrode and at an average distance of 1-50 mm from the working electrode (101)

The average distance may be 1-50 mm, 1-300 mm, 1-20 mm, 1-10 mm, 1-5 mm, 5-30 mm, 10-20 or 5-10 mm from the counter electrode and the working electrode respectively.

Thereby reducing the uncompensated solution resistance for the three-electrode system composed of porous electrodes enhancing the charge transfer of the system.

The device may further comprise an ion-selective membrane arranged between the working electrode and the counter electrode in the housing.

Due to the presence of the ion-selective membrane, at least some reaction products formed at the counter electrode may be stopped by the ion-selective membrane from reaching the working electrode, such as H₂O₂, other reactive oxygen species, or reactive redox species produced by impurities through electrocatalysis e.g. enzymes. For example an ion-exchange membrane such as sulfonated tetrafluoroethylene, poly(styrene sulfonate) would act to protect the fluid components in the working electrode compartment from those that may be present in the counter electrode compartment as described above while still permitting charge carriers to permeate the membrane and complete the electrical circuit.

At least a portion of a surface of the counter electrode may be provided with the same polyelectrolytic coating as the working electrode.

If both the working electrode and the counter electrode are provided with a pH-responsive polyelectrolytic coating, a fast switch can be made between the electrodes. In a first run the working electrode is used as the working electrode and the counter electrode is used as the counter electrode. In a second run, the counter electrode is used and the working electrode and the working electrode is used as the counter electrode.

The device described above may comprise two connected chambers, one chamber for the working electrode and one chamber for the counter electrode, separated by an ion-permeable membrane.

Separate chambers facilitate exchanging parts of the device when required. It permits optimizing the void space for the analyte of the active material.

Two connected chambers that lock together by an attachment mechanism may facilitate inserting the electrodes within the device.

According to a second aspect there is provided a system for separating an analyte from other components in an electrolytic solution. The system comprises the device described above, an arrangement for applying a potential difference between the working electrode and the counter electrode, a flow system arranged to supply the electrolytic solution to the housing at the solution inlet, and a solution collection system arranged at the solution outlet of the housing for collecting solution and analyte exiting the device through the solution outlet.

The arrangement for applying a potential difference may be a potentiostat controlling the voltage difference between the working electrode and the reference electrode by supplying a current through the counter electrode. The arrangement may be a three electrode system or it may omit the reference electrode, making the arrangement a two electrode system with only a working electrode and a counter electrode.

The purpose of the reference electrode is to have an inert and well known reference point where a minimal current passes through the reference electrode but where the a potential difference between the working electrode can be reliably specified, a voltage of 1 V is always corresponds to ˜1 V and can be compared to 1 V in separate electrochemical experiments, literature values and standard electrode potential tables. For instance, with an AgCl reference electrode by applying-1 V to a stainless steel working electrode oxygen reduction reaction will occur resulting in consumption of protons on the working electrode interface raising the surface pH as a result.

If the reference electrode is omitted in the device and only a working electrode and a counter electrode is used the well characterized reference point is lost. Applying-1 V cannot easily be compared to other literature values for standard potentials. The counter electrode may undergo permanent or temporary changes which changes its electrocatalytical properties causing a shift in the potential difference that is required to produce a sufficiently strong pH gradient to trigger analyte elution.

In all embodiments of the device, a potential is applied between the working electrode and the counter electrode.

In the method for using the device the specific voltage value used refers to the potential difference between the working electrode and the reference electrode.

For a well-established electrochemical process where the counter electrode is characterized and known to be relatively inert with little drift in electrochemical properties over many cycles 10 to 1000 cycles, the reference electrode may be a redundant part. Thereby a simplified two electrode system could be used.

With a two electrode system the potentiostat may also become redundant since a potential could in principle be applied by simply connecting a DC battery as power source for the device.

The simplification of the system having two electrode parts comprises a simpler system where a battery with appropriate voltage window 0 to 1.5 V could be used to supply power to the electrodes of the device.

At the solution outlet, the solution coming out of the outlet is collected. The solution exiting the device may be collected in fractions. This solution comprises fractions of different components. Depending on the voltage applied, different analytes are eluted from the device.

The system may further comprise a solution analysis device arranged to analyse the content of the solution collected at the solution outlet.

The solution analysis device may for example comprise a UV analyser, a fluorescence detection analyser, or analysis may be performed by sodium dodecyl sulphate-polyacrylamide gel electrophoresis (SDS-PAGE), Enzyme Linked Immunosorbent Assay (ELISA), Real-time polymerase chain reaction (qPCR) or other analytics assays.

According to a third aspect, there is provided a method of separating an analyte from other components in an electrolytic solution. The method comprises to provide a system described above, provide an electrolytic solution comprising an analyte to be separated from other components in the electrolytic solution, supply the electrolytic solution comprising the analyte to the housing at the solution inlet, allowing the solution to flow from the inlet to the outlet such that the analyte is captured by the polyelectrolytic coating arranged on the working electrode, apply a potential difference between the working electrode and the counter electrode, thereby releasing the analyte from the polyelectrolytic coating and eluting the analyte from the working electrode, and to collect the solution comprising the analyte exiting through the solution outlet.

Elution, release, of bound analyte to the working electrode can be achieved by applying a potential difference, an electrochemical signal, that alters the surface pH and thereby the intermolecular interactions between the polyelectrolytic coating on the working electrode and the bound analyte.

The electrochemical potential produces a local pH gradient that disrupts a specific interaction between resulting in elution without changing the solution pH of the device.

In one method favourable binding conditions is achieved at neutral pH to slightly basic pH e.g. pH 7-8, by introduction of the analyte to a functionalized polyelectrolytic coating, where immediate binding between a highly specific ligands occurs.

By changing the ionic concentration of the electrolytic solution you can change the pKa of the polyelectrolytic coating, and thereby change the conditions for attractive and repulsive interactions between the analyte and the polyelectrolytic coating. This can be used to change the pH at which analytes spontaneously bind to the polyelectrolytic coating. For instance, at lower total salt concentrations the pKa of polyacidic coatings composed of carboxylic acids is raised to neutral pH, allowing capture of biomolecules to occur at neutral pH instead of a slightly acidic pH.

In one method the total salt concentration and buffer capacity of the electrolytic solution is low, enabling highly sensitive switching of the interface pH by application of very small currents (<100 μA) and potentials (±100 mV).

The method may comprise a step before supplying the electrolytic solution comprising the analyte to the device of running a buffer through the device, the running buffer having a pH between pH 5 to pH 7.5.

The pH is chosen based on the preferred choice of buffer for the analyte in combination with at which specific pH and solution composition the analyte binds spontaneously to the electrode.

The buffer is used as a background buffer to equilibrate the system at the selected pH and salt concentration where separation is to be conducted. The running buffer does not bind to the working electrode. Analyte interactions are favoured, resulting in binding of said analyte to the working electrode of the device.

The running buffer used is a buffer that does not comprise any chemical species that may be harmful to the analyte, cause degradation, be of environmental concern or add significantly to the process cost. Examples of such running buffers and concentrations of such buffers are imidazole 500 mM, highly acidic buffers e.g. pH 2-3 100 mM acetic acid buffers or 0.1 M glycine×HCl PH 2-3, sodium hydroxide 0.5 M, organic surfactants and organic solvents such as ethylene glycol, glycerol, PEG, amino acids, sodium alkyl sulphate.

The electrolytic solution comprising the analyte may be supplied at the solution inlet at a flow rate of 0 mL/min to 10 L/min.

Thereby providing sufficient residence time for efficient binding of analyte to the polyelectrolytic coating of the working electrode, while allowing the entire analyte sample to flow through the device to maximize uptake.

When applying a potential difference between the working electrode and the counter electrode that mediates release of the analyte from the polyelectrolytic coating the flow rate the running buffer flow rate may be any value from stagnant 0 mL/min to 10 L/min.

The preferable flow rate value for applying a potential difference is a relatively low value ranging from 0 to 2 mL/min. A suitable flow rate range while performing electrochemical potential is 0.01 to 0.6 mL/min.

The running buffer flow rate may be varied throughout the duration of the application of the potential.

Thereby facilitating the establishment of an electrochemical pH gradient that sufficiently shifts the surface pH value within the polyelectrolytic coating to trigger release of the analyte.

Before the electrochemical potential is turned off, from 5 to 15 seconds before, the flow rate of the running buffer is preferably raised to at least five times as high. For instance, if the flow rate is 0.1 mL/min while performing the electrochemical elution it is desirable to increase the flow rate temporarily to 0.5 mL/min before the signal is turned off.

Thereby the analyte is rapidly transported away from the working electrode surface, preventing rebinding of the analyte to the surface of the working electrode when the pH at the surface of the working electrode returns to the stable running buffer pH value.

The total range for eluting the analyte from the working electrode, may be a running buffer flow rate of 0.01 mL/min to 10 mL/min.

Thereby permitting fast evacuation of the analyte.

After the step of allowing the electrolytic solution to flow from the inlet to the outlet such that the analyte is captured by the polyelectrolytic coating arranged on the working electrode, the device may be rinsed to remove unbound analytes and other components in the solution from the interior volume of the device.

The step of applying a potential difference between the working electrode and the counter electrode, thereby eluting the analyte from the working electrode, may comprise applying a constant potential difference over time where the duration may be 1 second to 3600 seconds. It may be 1-10 seconds, 10 to 30 seconds, 30-60 seconds, 60-120 seconds, or 120 to 300 seconds, or 300 to 600 seconds, it may also be 600 to 3600 seconds long.

Thereby a pH gradient is established. The extent of the pH gradient is primarily determined by (i) the buffer capacity of the solution, which counteracts the electrochemical reaction that alters the surface pH, and (ii) the magnitude of the electrochemical potential which determines the rate of the electrochemical reaction on the surface, (iii) the concentration of electroactive species i.e. proton accepting, or proton donating species, (iv) the flow rate renewal of buffer and mass-transfer properties diffusion and convection through the device.

The constant potential difference applied may be a positive or negative potential with a magnitude between 0 V and 1.5 V.

The potential difference is applied in the presence of a redox species that can produce or consume protons to change the pH.

The potential is positive if the intention is to lower the pH on the surface of the electrode, the potential is negative if the intention is to increase the pH on the surface.

Applying a potential difference between the working electrode and the counter electrode, thereby eluting the analyte from the working electrode, may comprise varying the potential difference continuously between two potential values over time where the duration may be 1 second to 600 seconds. It may be 1-10 seconds, 10 to 30 seconds, 30-60 seconds, 60-120 seconds, or 120 to 300 seconds, or 300 to 600 seconds, it may also be 600 to 3600 seconds long.

By application of a variable electrochemical potential a variable electrochemical potential will establish a variable pH gradient where in addition to the above-mentioned effects the rate of potential change will affect the extension of the pH gradient and result in a temporal variation in the change of the surface pH. The variable potential may be a step-wise increasing potential difference, resulting in a step-increase in pH that produces net-electrostatic repulsion between the electrode and the analyte. The potential difference applied may be continuously varied between two positive or negative voltage values within the magnitude range of 0 V to 1.5 V and for a duration between 1 to 3600 seconds.

The duration may be 1 second to 600 seconds. It may be 1-10 seconds, 10 to 30 seconds, 30-60 seconds, 60-120 seconds, or 120 to 300 seconds, or 300 to 600 seconds, it may also be 600 to 3600 seconds long.

The selected potential window between which the voltage is varied may vary depending on which redox active species is present in the electrolytic solution that alters the electrode surface pH. The local pH on the surface may be measured using analytical techniques to connect the potential window and voltage values used with the actual pH produced on the surface.

Providing a variable potential difference and providing a constant potential difference may be combined for eluting an analyte. For example, a varied potential may be used initially, followed by a constant potential difference.

By varying the potential difference continuously and at different speeds, as compared to just applying a potential suddenly, a separation with higher resolution can be obtained and you can gradually separate analytes as the interaction with the polyelectrolytic coating changes.

By varying the potential, it is possible to selectively desorb certain analytes such as virus capsids filled with genetic material, from empty virus capsids or partially empty virus capsids, and from host-cell proteins.

The resolution for separation of analytes by their difference in isoelectric point may be as low as 0.4 pH units, which is the isoelectric point difference for separation between filled and empty virus capsids.

By application of a potential where biomolecules with a certain isoelectric point are unbound from the polymer surface, the remaining analyte molecules are separated from biomolecule impurities and other impurities that may be present in the electrolytic solution.

By use of an electrochemical signal, it is possible to instantaneously release all/a majority of analytes bound to the working electrode, increasing the concentration of the separated pure analyte sample from a dilute sample.

With one electrochemical signal it is then possible to increase the concentration of the analyte sample by at least 20 times.

Binding of analytes to the surface of the working electrode can be highly efficient, with low loss of analyte.

Sample retention, the amount of bound analyte that is recovered by electrochemical signal was measured to be up to but not limited to 94%.

With use of the device an in-line up-concentration with sample retention and buffer exchange was achieved with highly competitive figures compared to off-line treatments like centrifugation and dialysis.

According to a fourth aspect there is provided method of concentrating an analyte in an electrolytic solution, comprising: providing a system as described above, providing an electrolytic solution comprising an analyte to be concentrated, supplying the electrolytic solution comprising the analyte to the housing at the solution inlet, allowing the solution to flow from the inlet to the outlet such that the analyte is captured by the polyelectrolytic coating arranged on the working electrode, applying a potential difference between the working electrode and the counter electrode of −1.5 to −0.5V, thereby immediately releasing the analyte from the polyelectrolytic coating, eluting the concentrated analyte from the working electrode, collecting solution comprising the concentrated analyte exiting through the solution outlet.

A potential is applied between the working electrode and the counter electrode. A specific voltage value used, as above, refers to the potential difference between the working electrode and a reference electrode (when used).

The concentration method produces a sample of the analyte which does not need additional post-processing operation off-line by centrifugation or dialysis which may result in substantial yield loss of the analyte product furthermore the analyte sample does not contain elution chemicals like high concentration of salt, surfactants, organic chemicals, or highly acidic or basic pH solution which would contaminate the analyte sample.

The methods may further comprise a step of cleaning the device after the elution step, by applying a potential difference between the working electrode and counter electrode, which potential difference is higher that the potential difference used during the elution.

Cleaning after elution may be performed by electrochemical cleaning of the working electrode to remove any eventual unbound biomolecules bound to the working electrode, by application of a slightly higher potential than required to achieve a high temporary surface pH resulting cleaning of the surface of the working electrode without disassembly.

Alternatively, cleaning may be performed by flowing cleaning solution through the device.

### BRIEF DESCRIPTION OF THE DRAWINGS

FIG. 1a shows a device for separating an analyte from other components in an electrolytic solution. FIG. 1b shows the same device in a cross-sectional view.

FIG. 1c shows a similar device as in FIG. 1a for separating an analyte from other components in an electrolytic solution. FIG. 1d shows the same device in a cross-sectional view.

FIG. 2a shows a system for separating an analyte from other components in an electrolytic solution, wherein the system comprises the device in FIG. 1a.

FIG. 2b shows a system for separating an analyte from other components in an electrolytic solution, wherein the system comprises the device in FIG. 1c.

FIGS. 3a and 3b show an embodiment of the device in FIG. 1a containing two connected chambers, one chamber for the working electrode and one chamber for the counter electrode. FIG. 3c shows the same device in a cross-sectional view.

FIG. 4a shows an embodiment of the device, wherein the working electrode comprises a hollow cylinder. Liquid is arranged to pass through the mantle surface of the working electrode and cross the centrum axis of the cylinder shaped electrode. FIG. 4b shows the same device in a cross-sectional view taken from top to bottom. FIG. 4c shows the same device in a cross-sectional view taken through the mid-section.

FIG. 5a illustrates how a polyelectrolytic coating is arranged on a working electrode and how such a functionalized working electrode catches and releases proteins in a solution upon application of a potential difference between the working electrode and a counter electrode.

FIG. 5b is a close-up illustration of how a polyelectrolytic coating is arranged on a porous working electrode within the device and how such a functionalized working electrode catches and releases proteins in a solution upon application of a potential difference between the working electrode and a counter electrode.

FIG. 6 shows a chromatogram where human serum set to pH 5 is captured and separated by selective elution using electrochemical signals.

FIG. 7 shows a QCMD sensorgram of a PAA functionalized stainless-steel surface where BSA is captured and released by electrochemistry with stepwise increase in the potential magnitude as indicated in the figure.

FIG. 8 shows a chromatogram showing electrochemical elution of a protein, BSA, captured in the polyelectrolytic coating on the working electrode.

FIG. 9 shows a potentiostat readout from a cyclic voltammetry (CV) scan applied (variable potential between 0 V to −1 V at a rate of 100 mV/s) for electrochemical elution of the protein as shown in FIG. 8 denoted Signal 1 in FIG. 8, providing one example of how electrochemical elution may be accomplished.

FIG. 10 shows a potentiostat readout from a chronoamperometry (CA) scan (constant potential applied at −1 V for 300 s) denoted Signal 2 in FIG. 8, providing one example of how electrochemical elution may be accomplished.

FIG. 11 shows a chromatogram showing pH step elution of a protein, BSA, captured in the polyelectrolytic coating on the working electrode.

FIG. 12 shows a chromatogram showing a solution pH gradient separation of a mixture of three proteins (bovine serum albumin (BSA), lysozyme (LYS), and Lactoferrin (LAC)) captured in the polyelectrolytic coating.

FIG. 13 shows a chromatogram where human serum diluted with PBS and set to pH 5 is separated into discrete fractions using electrochemical signals in discrete steps as indicated.

FIG. 14 shows a chromatogram where human serum is separated with electrochemical elution when diluted with PBS and set to pH 5.

FIG. 15 shows an image of a stained SDS-PAGE gel where eluted samples of human serum proteins by pH gradient.

FIG. 16 shows a stained SDS-PAGE gel showing eluted human serum proteins by electrochemical signals.

FIG. 17 shows chromatogram where a sample of human serum is mixed with 1/10×PBS at pH 7 and is separated into discrete fractions using electrochemical signals. FIG. 18 shows electrochemical signals used to elute the discrete samples of human serum proteins of FIG. 16 between −0.4V to −1.2 V.

FIG. 19 shows a chromatogram where a sample of human serum is mixed with 1/10×PBS at pH 7 and is separated by a pH and salt gradient (0.1M NaOH).

FIG. 20 shows an image of an SDS-PAGE gel containing samples collected from electrochemically purified human serum proteins (as shown in FIG. 17).

FIG. 21 shows an image of an SDS-PAGE gel containing samples collected from human serum separated with pH gradients (as shown in FIG. 17 and FIG. 18.).

FIG. 22 shows a QCMD sensorgram of a PAA polymer brush which is converted into a brush with NTA-Me²⁺ functional groups where a recombinant protein with His-tags are immobilized by metal-ion affinity and released by imidazole elution and by electrochemical elution as indicated in the figure.

FIG. 23 shows a QCM sensorgram with frequency and dissipation signals monitored for a polyacidic polyelectrolytic coating, a brush, which is post-functionalized with Protein A and where antibodies are immobilized by specific binding interactions with Protein A on the surface and released by injecting a pH 2.3 solution.

FIG. 24 shows a QCM sensorgram with frequency and dissipation signals monitored for a polyacidic polyelectrolytic coating, a brush, which is post-functionalized with Protein A and where antibodies are immobilized to Protein A by specific interactions and released by application of positive electrochemical potentials, +0.6 V, in a 5 mM hydroquinone solution.

FIG. 25 shows a chromatogram wherein purified monoclonal antibodies (IGG) are captured using affinity interactions with a protein A functionalized polymer coating on a microporous stainless steel mesh, then eluted with solution pH change.

FIG. 26 shows a chromatogram wherein purified monoclonal antibodies are captured using affinity interactions, with a protein A functionalized polymer coating on a microporous stainless steel mesh, then eluted using basic electrochemical signals, by negative potentials.

FIG. 27 shows cyclic voltammetry scans used to generate basic elution by scanning a negative potential window −0.7 V to −0.8 V that increases the surface pH that breaks the protein A antibody binding to release antibodies from the surface.

FIG. 28 shows a chromatogram where a sample of purified monoclonal antibodies is captured using affinity interactions with a protein A functionalized polymer coating on a microporous stainless-steel electrode followed by elution by acidic electrochemical signal, positive potential in the presence of a redox probe 5 mM hydroquinone in the buffer.

FIG. 29 shows cyclic voltammetry scans used to generate a local acidic pH gradient on the surface by cycling in a positive potential window +0.7 V to +0.8V, resulting in release of antibodies from the Protein A coating (see the chromatogram FIG. 28)

FIG. 30 shows a SDS-PAGE gel comparing bands from elution of monoclonal antibodies by basic electrochemical elution (C6-C8) with pH solution release (A3-A5).

FIG. 31 shows an SDS-PAGE gel with flow-through elution of monoclonal antibodies with electrochemical elution with lanes indicated in the corresponding chromatogram FIG. 28.

FIG. 32 shows a chromatogram where monoclonal antibodies are captured from clarified cell culture harvest following collection of samples by electrochemical elution of purified antibodies (wells F1, F2, F4, G1-G4).

FIG. 33 shows an SDS-PAGE gel indicating presence of purified monoclonal antibodies in lanes (F1, F2, F4, G1-G4) from samples produced in as described for FIG. 32.

FIG. 34 is a chromatogram showing enrichment of a dilute protein sample where 6% of the protein sample flows through the electrode without binding, and 94% of the sample is captured and subsequently released as an enriched/concentrated sample by application of an electrochemical signal.

FIG. 35 shows a cyclic voltammetry scan used to elute the proteins captured on the working electrode under the conditions mentioned for FIG. 34. The potential was varied between −1 V to −0.75 V for 120 s.

FIG. 36 is a chromatogram showing purification using electrochemical signals of AAV (Adeno-associated virus) filled virus capsids from filtered cell supernatant containing, empty viral vectors and host cell proteins.

FIG. 37 shows an analysis of samples from the purification in FIG. 36. FIG. 37 shows gene count determined by qPCR, capsid count by ELISA for detection of AAV capsids, and a calculation of the ratio of filled over empty particles for each sample collected.

FIG. 38 is a chromatogram showing purification using solution pH change for elution of AAV filled virus capsids from filtered cell supernatant containing empty viral capsids and host cell proteins.

FIG. 39 shows an analysis of samples from the purification shown in FIG. 38. FIG. 39 showing gene count determined by qPCR, capsid count by ELISA for detection of AAV capsids, and a calculation of the ratio of filled over empty particles for each sample collected.

FIG. 40 shows liposomes spontaneously captured to a polyelectrolytic coated electrode in multiple layers with high binding capacity and subsequently released in a gentle manner, where the liposomes are proven to be intact by subsequent measurement of the particle size, compared before and after.

### DETAILED DESCRIPTION

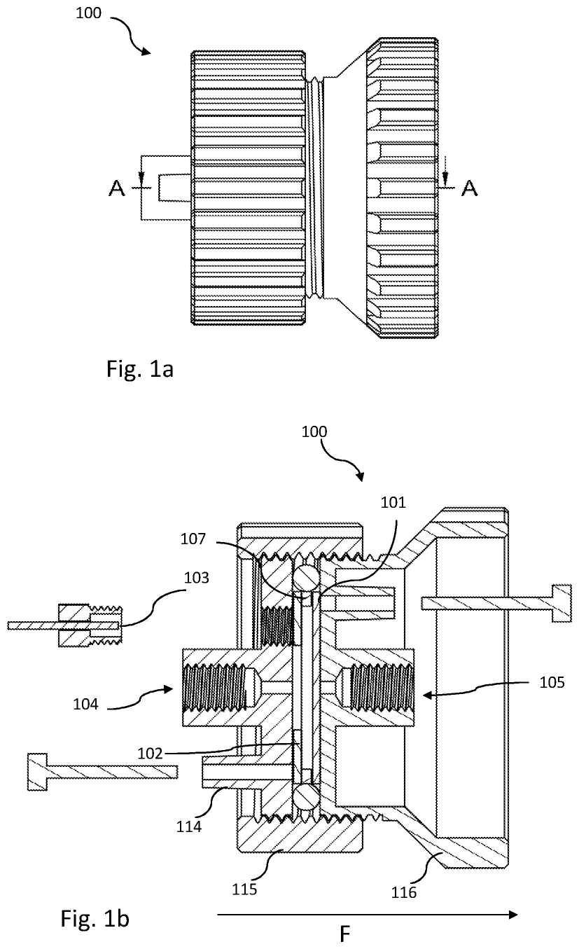

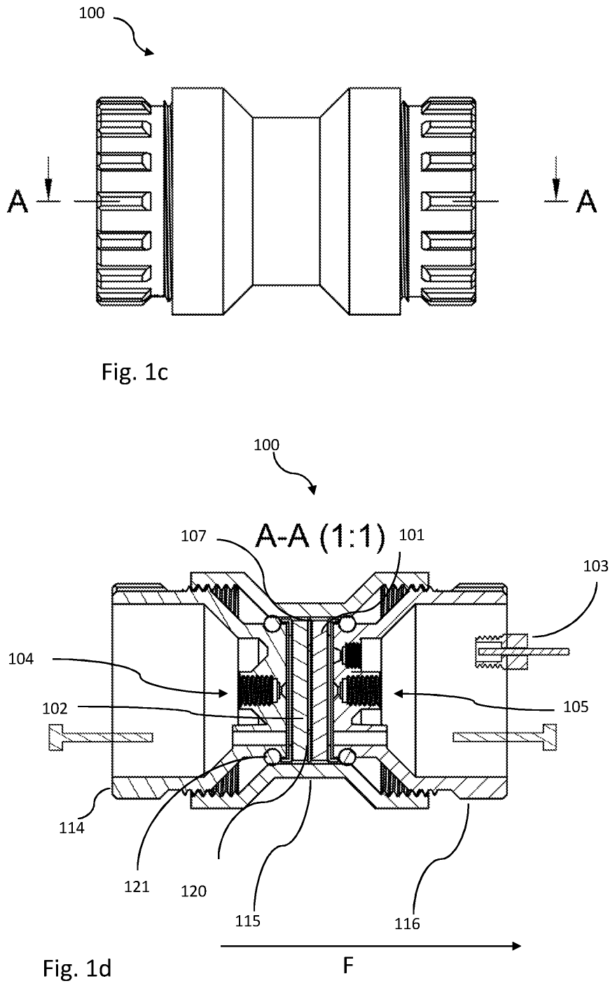

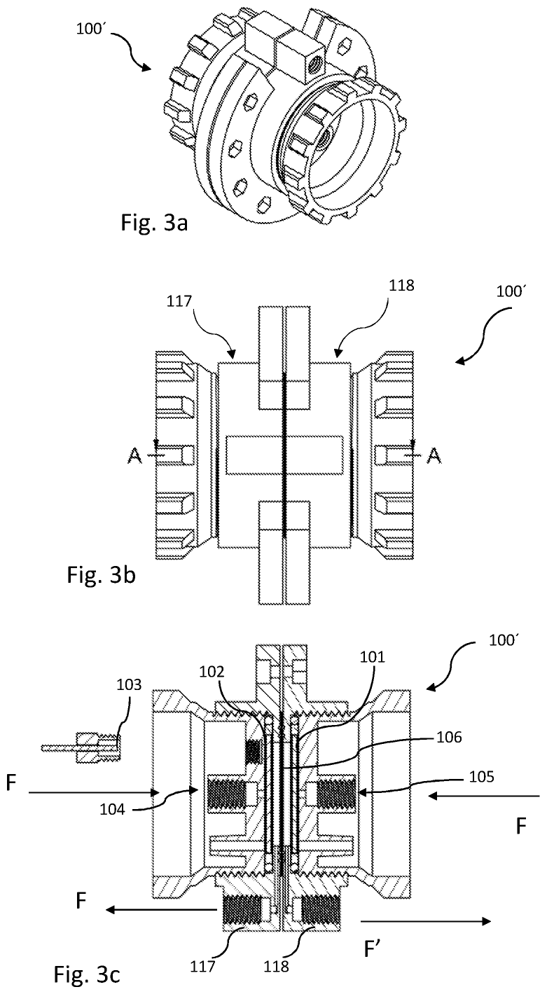

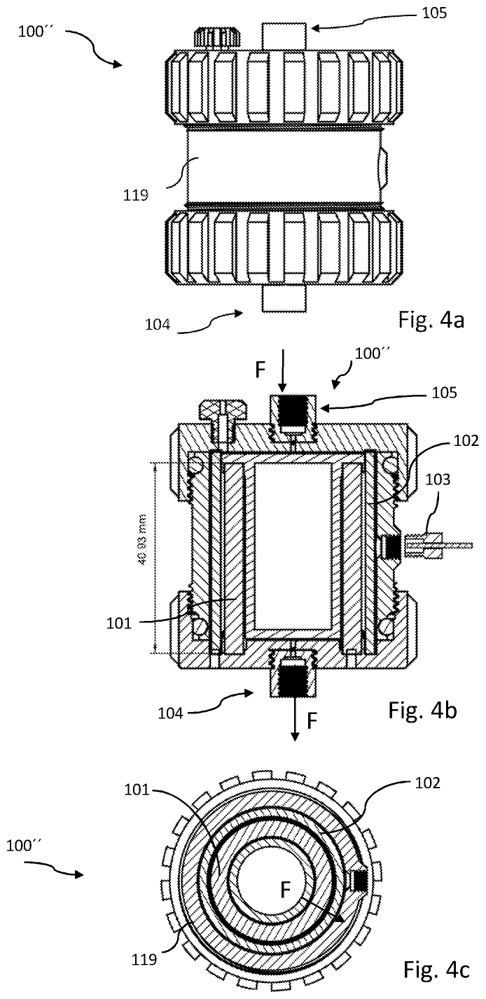

Below is described a device, system and method for non-invasive, separation and concentration of analytes, such as biomolecules, from other components in an electrolytic solution. FIG. 1a and FIG. 1c shows such a device 100. FIG. 1b and FIG. 1d shows the same device 100 in a cross-sectional view. In FIG. 1 b part 116 and 114 squeezes the o-ring gasket part 121 which seals the device permitting flow through the inlet 104 and outlet 105. The combined thickness of the electrodes plus spacers are limited by the diameter of the o-ring/or thickness of gasket. An alternative design of the device is shown in FIG. 1d where the o-ring is wrapped around part 114 and 116 thicker electrodes can be used where part 107 can be extended in suitable length to accommodate thick electrodes. In FIGS. 3a-3c is shown another embodiment of the device 100′ and in FIGS. 4a and 4b is shown yet an embodiment of the device 100″.

The device 100, 100′, 100″ comprises a housing 114, 115, 116, 117, 118, 119 provided with a solution inlet 104 and a solution outlet 105. The housing may be in one part. Alternatively, the housing may comprise two or more parts connected/connectable to each other. In FIGS. 3a, 3b and 3c is illustrated a device 100′ with a housing comprising two connected parts 117, 118. In FIG. 3b is illustrated a housing comprising three connected parts 114, 115, 116. The housing or the housing parts may for example be 3D printed or injection-moulded in e.g. plastics. One way to realize the device as described in FIG. 3 a b c is to make several connectable 3-D printed parts with threads to connect and with o-ring as packings for sealing. A device that assembles with separate parts may be advantageous to facilitate placement of electrodes within the device and connecting these with exterior electrode pins such that the electrodes can be connect to an electronic equipment like a potentiostat. By producing the device in several parts by 3D-printing it may be possible to directly integrate electrodes into the design, or to post-process the parts to contain a conductive coating. However, the device may be produced by different methods in a single piece with moulded plastics and hermetically sealed parts and it may need to be in a sterile environment for industrial manufacture and with regulatory compliance. The entire polyelectrolytic coating process may be produced inside a hermetically sealed device.

A working electrode 101 is arranged in the housing in a space between the solution inlet 104 and the solution outlet 105, and arranged such that an electrolytic solution arranged to flow from the inlet to the outlet contacts at least a portion of the working electrode.

The working electrode 101 may be of any conductive material, such as carbon, a noble metal such as gold, a conducting oxide, a conductive plastic, stainless steel, aluminium, nickel metal foam (with micrometer sized pores) or a conducting polymer. The working electrode 101 may be of a solid material, of a porous material such as a mesh, a foam or a nano-hole array. A conductive sheet of mesh or foam or membrane may be stacked in multiple layers to reach a total electrode volume that satisfies the over-all analyte binding capacity required for a separation operation.

The electrode may be microporous or mesoporous, allowing a multi-scale hierarchical porous structure, which would allow a high surface area and thus a high analyte loading capacity. Using an electrode with a porous structure, an electrode with high surface area is obtained, thereby enabling high-capacity (several μg/cm²) immobilization of analytes. The porous electrode may have a porous surface with pores of about 1 μm. The pore size interval ranges from 500 to 10 um for foams, and for woven meshes 10 to 1 micrometers. In general an electrode with pore size between 0.5 to 2 micrometer is preferable.

The percentage of void volume may be in an interval between 50% to 99%. The material density can range between 0.05 to 1.5 g/cm3. The electroactive surface area per volume of electrode may range between 100 to 10 000 m2/m3. On the one hand a very high porosity increases convective mass-transfer of analyte to the surface, decreases the pressure gradient across the device. On the other hand a very high porosity eventually leads to too large pore sizes, loss of filtering effect, loss of surface area for capture, creates a mechanically fragile structure. Low porosity of the working electrode enables large surface area with very fine pores and a mechanically stable structure, but a too low porosity creates diffusion limited flow of analytes to the surface, increases risk of clogging, produces large pressure drop, A preferable working electrode has a 1 micrometer pore size, has a porosity of 95%, is very light with 0.05 g/cm3 and an electroactive surface area of at least 5000 m2/m3.

A working electrode with electroactive surface area of 5000 m2/3 functionalized with polyelectrolytic coating with a binding capacity 5000 ng/cm2 corresponds to a binding capacity of 250 mg/cm3 which is well beyond the binding capacity of a state of the art chromatography resin. The range of volumetric binding capacities of the working electrode may range between 5 to 500 mg/cm³.

The total binding capacity could be extended by for instance improving the polyelectrolytic coating thickness or other means of increasing the binding capacity per surface area, or by creating even higher electroactive surface area of the working electrode by for instance introducing a surface roughness.

A total area of the electrode surface may comprise from 50% to 97% of voids. With a porous electrode, the solution is filtered through a micrometer aperture. The density of the material can be between 0.05 to 1.5 g/cm³, and the surface area per volume unit of the porous electrode may range between hundreds to several thousands of square meters per cubic meter volume (100-10 000 m²/m³).

The pressure drop across the device when using microporous woven electrodes with 1 micrometer aperture and 0.1 mm thickness lies between 0.05 MPa to 0.3 MPa depending on the flow rate. The pressure drop across the device depends on the pore aperture, porosity, and the total thickness of the electrode. The working electrode 101 may be of any shape, such as a cylinder, which may be solid or hollow. The working electrode may be a plate of rectangular, circular, etc. shape. FIGS. 4a and 4b show a working electrode 101 being a hollow cylinder where flow passes through the working electrode in the radial direction. The flow is arranged to pass through the mantle surface of the working electrode and cross the centrum axis of the cylinder shaped electrode.

The working electrode 101 may be porous and arranged in the housing such that the electrolytic solution is allowed to flow from the solution inlet 104 through at least a portion of the working electrode 101 to the solution outlet 105, as illustrated in for example FIG. 1b.

A main direction of extension of the working electrode 101 may extend in a direction substantially perpendicular to a flow direction, F, from the solution inlet 104 to the solution outlet 105, such that flow may pass substantially vertically through the working electrode 101 structure. The working electrode 101 may span the entire flow path between the solution inlet 104 and solution outlet 105, as illustrated e.g. in FIG. 1b ensuring that there is a flow, F, of liquid through the working electrode 101.

A counter electrode 102 is arranged in the housing in a space between the solution inlet 104 and the solution outlet 105 at a distance from the working electrode, and arranged such that it is in electrical connection with the working electrode 101 via the electrolytic solution arranged to flow from the inlet to the outlet. The counter electrode 102 is arranged in the housing at a distance from the working electrode 101, such that there is non-contact between the electrodes, i.e. low risk of shortcut. A spacer 107 (see e.g. FIG. 1b) may be arranged to keep the working electrode 101 and the counter electrode 102 physically separated.

The effective surface area of the counter electrode 102 is preferably at least 2 times larger or 2-4 times larger than the effective surface area of the working electrode 101, to ensure sufficient capacity for the counter electrode to close the circuit without degradation for any given potential between −1.5 V to +1.5 V on the working electrode. The above mentioned size relationship may be especially valid if the electrode materials are the same or similar. In the situation of a electrodes composed of different materials the relationship could be different. In general, the counter electrode is larger than the working to ensure supply of sufficiently current capacity, electrostatic charge build-up+faradaic reactions, that a controlled voltage can be applied between the working electrode and the reference electrode. However, with different materials chemistries of the electrodes the counter electrode could be equal or even smaller with acceptable electrochemical elution properties. For instance, the counter electrode could be carbon-based with very high surface area with supercapacitor properties enabling large electrostatic charge build-up. It could be composed of titanium doped with platinum or ruthenium with high faradaic charge transfer properties.

The flow rate during the loading of analytes into the device may be as low as 0.01 mL/min for concentrated analyte samples, which allows time for all analytes to bind to the working electrode. The binding flow rate may as low as stagnant where the sample is first pumped into the device, the flow speed is set to zero, while as much as possible of the analyte sample is bound to the electrode. For low concentrated samples the flow rate can be higher 1-5 mL/min while still allowing most analytes to bind to the surface as they flow through the electrode. The current device tolerates flow speeds up to 10 mL/min. For scaled up models of the device the binding flow rate may be higher if optimization of the binding requires it.

The device may incorporate o-rings or gaskets 121 suitably arranged in all openings of the device to ensure that it is leak-proof and tolerates high flow speeds up to 10 mL/min and high pressures up to 0.6 MPa. The gasket or o-ring material may be composed of Nitrile, rubber, elastomer or silicone.

The plastic parts of the entire device 100 may be 3D-printed in waterproof plastic materials which are also tough and durable like PETG, PP, PEEK, Teflon and similar materials to ensure that it is water-proof and tolerates high pressure gradients during its operation.

The device could be manufactured to tolerate substantially higher pressure gradients if needed by use of a different manufacturing methods like injection moulding instead of Fused Deposition Modeling (FDM) 3D-printing.

The device may include a spacer 120 between the inlet 104 and the working electrode 101 that promotes mixing and turbulent flow of the sample prior to binding to the surface, enabling enhanced convective mass transfer to and from the surface of the working electrode.

The device may be manufactured to have such structures on its interior surfaces contacting the solution flowing through the device such that it increases convection, promoting turbulence, adding uptake of the analyte to the electrode by increased mass transfer to and from the electrode surface.

Reflux of sample that flows from the outlet 105 during the loading phase may be re-introduced through the inlet 104 back into the device to ensure complete binding of all available analytes.

FIGS. 3a-3c show an embodiment of the device 100′ where the housing 117, 118 consists of two connected parts, one for the working electrode 101 and one for the counter electrode 102.

The counter electrode 102 is preferably arranged close to the working electrode 101 (mm, micrometer or even nm distance). Further, the surface area of the counter electrode 102 should preferably be at least of the same size as that of the working electrode 101, and may be larger. The material of the working electrode may be a stainless steel alloy e.g. 316L, it may be carbon, it may be a noble metal e.g. gold or platinum, it may be a conductive polymer material, it may be made of aluminum, titanium or be a semiconductor. The material may be doped with a conductive element. The material may be a non-conductive polymer scaffold with very high surface area which is coated with a conductive film or foil to render it conductive. A conductive material with large surface area may be electroplated with a noble metal or with a metal film that has advantageous electrocatalytical properties.

Examples of non-conductive filter membrane structures that could be coated with a metal to create a high-porosity scaffold with large surface area are; polypropylene, nylon, cellulose, Teflon and polycarbonate membranes.

The shape of the electrode may be circular or rectangular. The electrode may be porous permitting flow through the electrode or it may be solid allowing flow to pass across the surface of the electrode. If the electrode is porous and if flow passes through the electrode the porosity may be between 50% and 97%. The internal surface area of the electrode may be between 100 m²/m³ to X1000 m²/m³. The working electrode 101 and/or the counter electrode 102 may be coated with a thin metal film, or doped with different metal elements, to provide different electrocatalytical properties, e.g. metal vapour deposition of a thin layer of for example but not limited to gold or platinum.

The device as shown in FIG. 3c enables separation of the working electrode 101 and the counter electrode 102 into two separate but connected compartments. The working and counter electrode are still connected through an ion-permeable membrane like Nafion (fluorinated polymers) or poly(styrene sulfonate). This is advantageous as it permits substantial lowering of the total void volume of the internal volume of the device that performs capture of the analyte. Removing the counter electrode from the internal volume where the analyte is introduced raises the capture efficiency of the device. For instance, if the counter electrode volume is 2 times larger than the working electrode the volumetric efficiency for capturing analyte becomes 3 times more effective if the working electrode is placed in a separated compartment from the counter electrode.

FIGS. 4a-c show an embodiment of the device where the working electrode (101) and counter electrode (102) have the shape of hollow cylinders permitting lateral flow through the electrode structure, meaning flow through the lateral surface of the cylinder, radial direction. Liquid is arranged to pass through the mantle surface of the electrodes and cross the centrum axis of the cylinder shaped electrodes.

A cylindrical arrangement of the working electrode (101) and counter electrode (102) material is advantageous if the electrode material is flexible, e.g. metal or conductive textile mesh or foam that is flexible and can be wrapped into a hollow cylinder shape. The electrode materials may also be manufactured in a cylindrical shape from a rigid but porous electrode structure.

One way to improve the binding capacity of a cylindrical shaped working electrode would be to prepare a larger sheet of electrode and wrap it multiple times to crease a hollow cylinder with thicker walls. Flow in the lateral direction provides effective mass transfer properties and achieves high binding rate and utilizes effectively the binding capacity of the working electrode.

The counter electrode 102 may be coated with a metal layer for instance by metal vapor deposition to improve the capacitive charging properties of the material, allowing a higher current density to pass through the counter electrode and thereby decreasing the required volume of counter electrode within the device.

At least a portion of a surface of the working electrode 101 is provided with a polyelectrolytic coating 111. If the electrode is porous, the polyelectrolytic coating may also extend into the pores of the electrode. A microporous electrode may constitute a filter before the solution reaches the polyelectrolytic coating. Using microporous electrodes coated with polyelectrolytic coatings offers not only a separation based on chemical interactions tuned by electrochemical signals. It also serves as a physical barrier that filters out larger objects, impurities, aggregates that may be present in the process flow, from the pure product that passes through the device.

The polyelectrolytic coating 111, a stimuli-responsive coating, being arranged to upon application of a potential difference between the working electrode 101 and the counter electrode 102 switch between a first, neutral, state, and second, charged, state, wherein in the first state an analyte is captured in the polyelectrolytic coating through non-electrostatic binding and in the second state a captured analyte is released/eluted from the polyelectrolytic coating through electrostatic repulsion.

The working electrode is regenerated by repelling/releasing/eluting the captured analyte when switched into its second, charged state, keeping the polyelectrolytic coating on the electrode surface. Thereby, the device can be used a repeated number of times using the same working electrode coated with the very same polyelectrolytic coating. No chemicals of environmental and health concern are needed for releasing/removing the captured analyte from the working electrode.

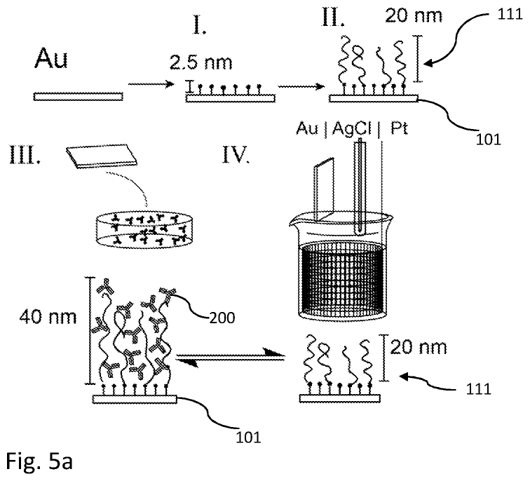

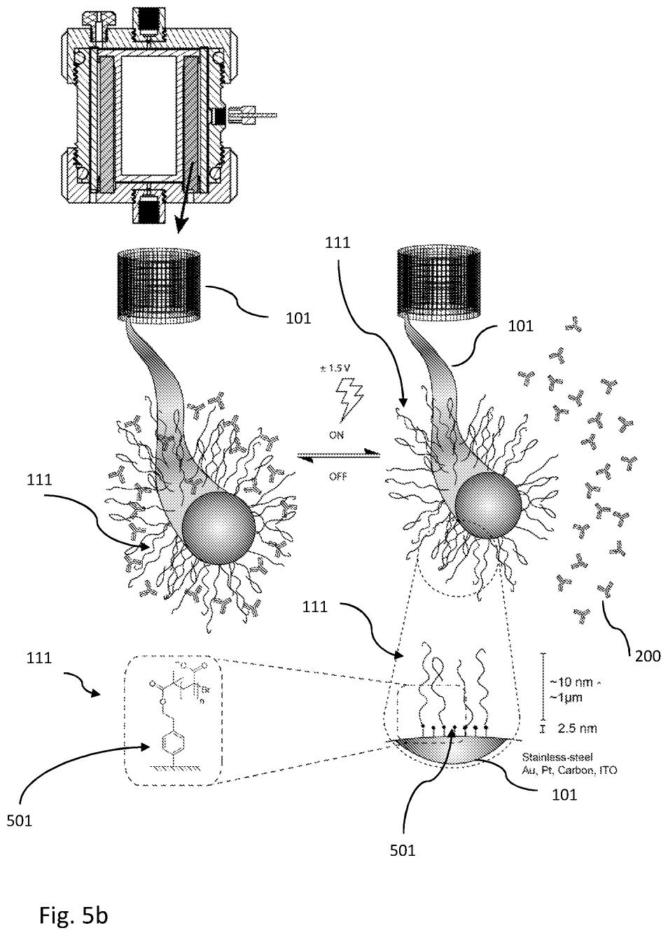

FIGS. 5a and 5b illustrate how a polyelectrolytic coating 111 is arranged on the surface of the working electrode 101 which in the example in FIG. 5b is a porous electrode surface composed of a woven mesh of stainless-steel thread. A close-up picture of the conductive thread shows how the surface of the working electrode 101 is functionalized with the polyelectrolytic coating anchored to the surface with an electrochemically stable aryl monolayer 501. FIG. 5b also shows how the polyelectrolytic coating may catch and release proteins 200 in a solution upon application of a potential difference between the working electrode 101 and a counter electrode 102.

The polyelectrolytic coating 111 may comprise a pH-responsive polymer covalently bound to the surface of the electrode through a monolayer of aryl bonds 501. The aryl bond is an electrochemically stable chemical anchor and enables tuneable release of the captured analytes. Due to these, electrochemically stable aryl bonds, the device and the polyelectrolytic coating on the working electrode surface can be reused a large number of times.

Through the application of a potential difference between the working electrode 101 and the counter electrode 102, a local microscale pH gradient is created that extends from the surface of the working electrode. The pH-sensitive/responsive polymer switches its state as a result of the local pH difference on the surface. The switch of the pH sensitive/responsive polymer results in either capture or release of the analyte from the surface of the electrode, which gives rise to a separation between the analyte and other components of the sample. The separation takes place due to a differing affinity towards the electrode for the analyte compared to other components in the sample solution. The difference in affinity comprises non-electrostatic intermolecular attractions, e.g. hydrogen bonding between the analyte and the polymer coated electrode. Further, it may be due to electrostatic attraction or repulsion.

The pH-responsive polymer may be a polymer comprising a carboxylic acid group. The pH-responsive polymer may be a polymer modified/functionalized to contain functional groups that are pH-responsive and have affinity for the analyte of interest. In some cases, the polymer is functionalized with a molecule with several functional groups that creates a “handle” for gripping specific analytes. Such handle could be a biological ligand molecule like Protein A if the analyte is a monoclonal antibody, or it could be a synthetically produced peptide with affinity to the target analyte. Such handles can be analyte-binding at one pH and be analyte-repellent in another state. The polymer may be selected based on knowledge of the analyte intended to be captured, what surface exposed binding pockets that may be present on the analyte, what specific interactions are present, and importantly how the strength of the binding is influenced by changes in the pH. The design and chemical modifications of the polymer is adjusted such that the analyte has a specific but pH dependent interaction with the side groups of the polymer.

The polyelectrolytic coating may be in the form of a polyelectrolyte brush, a film, a gel or layer. A thickness of such polyelectrolytic coating may be any value between a very thin coating on the nanoscale (˜1 nm) up to micrometers (˜1 μm). On the one hand a thin coating results in a low capacity for analyte immobilization per surface area of electrode but offers efficient switching of the entire coating already at low voltages (˜0.1 V). On the other hand, a thick microscale coating allows for a large quantity of analyte to be stored per surface area unit of the electrode surface but requires stronger electrochemical signals for efficient switching of the entire coating (˜1.0 V).

As is illustrated in FIGS. 3b and 3c, the device 100′ may be provided with an ion-selective membrane 106 arranged between the working electrode 101 and the counter electrode 101 in the housing 117, 118. Thereby, at least some reaction products, such as H₂O₂, formed at the counter electrode 102 may be stopped by the ion-selective membrane from reaching the working electrode 102.

When an electrochemical signal is applied to the working electrode 101 to change the pH on its surface, other electrochemical reactions may occur on the counter electrode 102 that produces an opposite pH change on the counter electrode surface. If the working and counter electrode surfaces are separated by a small volume of liquid, there may be mixing with the liquids and unwanted neutralization of the pH effect on the working electrode. The ion-exchange membrane 106 of FIGS. 3b and 3c may then be used to confine temporary undesired pH effects on the counter electrode 101 and thereby prevent undesired interference with the pH change that occurs on the working electrode 101. At least a portion of a surface of the counter electrode 102 may be provided with the same polyelectrolytic coating 111 as the working electrode 101. Thereby, a fast switch can be made between the electrodes 101, 102, and the electrodes are interchangeable. In a first run the working electrode 101 may be used as the working electrode and the counter electrode 102 is used as the counter electrode. In a second run, the counter electrode 102 is used as the working electrode and the working electrode 101 is used as the counter electrode. The working electrode 101 and counter electrode 102 may then be made of identical material and identical size, which allows switching between the electrodes as working and counter electrodes, thereby permitting rapid cycling and utilization of both for analyte separation.

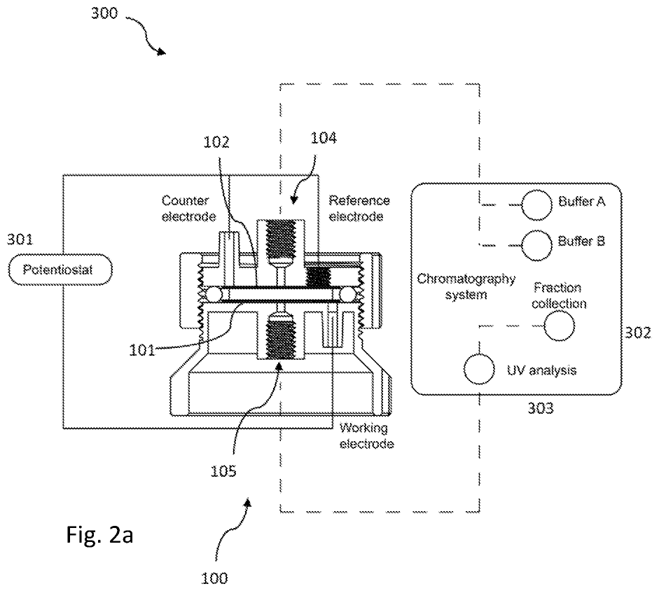

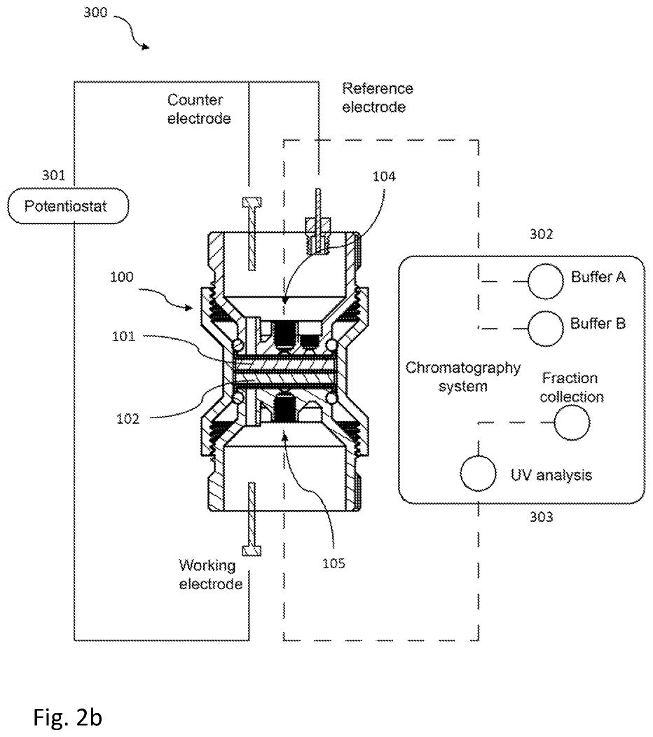

FIG. 2 Shows a system 300 for separating an analyte 200 from other components in an electrolytic solution, wherein the system 300 comprises the device 100, 100′, 100″ described above. The system 300 also comprises an arrangement 301 for applying a potential difference between the working electrode 101 and the counter electrode 102. Such a system 301 may be a potentiostat. A flow system is arranged to supply the electrolytic solution to the housing 114, 115, 116, 117, 118, 119 at the solution inlet 104, and a solution collection system 302 is arranged at the solution outlet 105 of the housing for collecting solution and analyte 200 exiting the device through the solution outlet 105. The solution exiting the device may be collected in fractions. This solution comprises fractions of different components. Depending on the voltage applied, different analytes are eluted from the device.

The flow system and the solution collection system may be a standard set-up of a traditional chromatography system, wherein the present device replaces the colon of a chromatographic system and further an arrangement for applying a potential difference to working electrode and counter electrode is added.

The system 300 may further comprise a solution analysis device 303, which may be a UV analyser, arranged to analyse the content of the solution collected at the solution outlet 105. The solution analysis device may be arranged to identify different analytes in the solution or fractions of analytes. The solution analysis device may be part of a standard chromatography system.

When an electrolytic solution comprising the analyte 200 has been provided to the housing at the solution inlet 104, and the analyte has been captured by the polyelectrolytic coating 111 arranged on the working electrode 101, a potential difference, an electrochemical signal, is applied between the working electrode 101 and the counter electrode 102, which alters the surface pH and produces a local pH gradient that disrupts the intermolecular interactions between the polyelectrolytic coating 111 on the working electrode 101 and the bound analyte 200, which can be collected at the solution outlet 105. This results in an elution of the analyte 200 without changing the solution pH of electrolytic solution.

The flow rate used for releasing analytes from the device should be high enough such that the analytes are transported from the working electrode by convection. The flow rate should be low enough so that the pH gradient can establish itself in the microenvironment without the flow rinsing away the pH gradient. With the specific design presented in FIG. 1b the range of flow rates where a trade-off between clearance of released analytes and efficient electrochemistry was found between: 0.01 mL/min and 5 mL/min. Depending on the specific design of the device, and the choice of working electrode properties the optimal flow rate window may be subject to change.

The electrolytic solution may comprise 1 mM to 1 M salt ions. The total salt concentration, the ionic strength, will influence the pKa of the polyelectrolytic coating. A high salt concentration leads to high pKa and a low salt concentration leads to low pKa, changing the pivot point between the first (neutral) stage and the second (charged state)—meaning the pH at which point the polyelectrolytic coating is analyte binding and repelling. To enable electrochemical reactions the electrolytic solution also comprises redox-active species.

By changing the ionic concentration of the electrolytic solution you can change the interaction between the analyte and the polyelectrolytic coating. This can be used to change the pH at which analytes spontaneously bind to the polyelectrolytic coating.

In one example the total salt concentration and buffer capacity of the electrolytic solution is low, enabling highly sensitive switching of the interface pH by application of very small currents (<100 μA) and potentials (±100 mV).

Before supplying the electrolytic solution comprising the analyte 200 to the device 100, 100′, 100″ a buffer may be run through the device. The running buffer is used as a background buffer to equilibrate the system at the selected pH and salt concentration where separation is to be conducted. The running buffer does not bind to the working electrode 101. Analyte interactions are favoured, resulting in binding of said analyte to the working electrode of the device.

The running buffer may for example be set to pH 5 to obtain favourable binding conditions by non-electrostatic attractions e.g. hydrogen bonding to the analyte entity to the predominantly neutral polyelectrolytic coating of the working electrode.

After the step of allowing the electrolytic solution to flow from the inlet to the outlet such that the analyte is captured by the polyelectrolytic coating arranged on the working electrode, the device may be rinsed to remove unbound analytes and other components in the solution from the interior volume of the device.

This can be seen as a decrease and stabilization of the in-line UV signal, when analysing the solution exiting the device.

To elute the analyte from the polyelectrolytic coating 111 of the working electrode a constant potential difference may be applied between the working electrode 101 and reference electrode 103.

Thereby a pH gradient is established. The extent of the pH gradient is primarily determined by (i) the buffer capacity of the solution that counteracts the electrochemical reaction that alters the surface pH, and (ii) the magnitude of the electrochemical potential which determines the rate of the electrochemical reaction on the surface.

A reductive continuous potential (chronoamperometry) applied may be between −0.1 V to −1.5V. An oxidative continuous potential may be +0.1 to +1.5 V. The potential used depends on the polyelectrolytic coating, the analyte, the electrodes used etc. In one example, for reductive potential, that is, to raise the pH, a potential difference of −0.3 V to −1.5 V may be used. For oxidative potential (with hydroquinone as redox-active species) to raise the pH and when the electrode is coated with a metal such as gold or platinum) a potential difference of +0.25 V to +1.5 V may be used.

Alternatively, to elute the analyte from the polyelectrolytic coating 111 of the working electrode a continuously varied potential difference (cyclic voltammetry), electrochemical potential, may be applied between the working electrode 101 and reference electrode 103. A variable electrochemical potential will establish a variable pH gradient where the rate of potential change will affect the extension of the pH gradient and result in a temporal variation in the change of the surface pH. The variable potential may be a step-wise increasing potential difference, resulting in a step-increase in pH that produces net-electrostatic repulsion between the electrode and the analyte.

Providing a variable potential difference and providing a constant potential difference may be combined for eluting an analyte. For example, a varied potential may be used initially, followed by a constant potential difference.

By varying the potential difference continuously and at different speeds, as compared to just applying a potential suddenly, a separation with higher resolution can be obtained and you can gradually separate analytes as the interaction with the polyelectrolytic coating changes.

Adjustment of the time, duration of the electrochemical signals will affect the extent to which biomolecules are exposed to the local pH gradient. The duration of the signal can be selected based on the relative pH sensitivity profile of the target analyte and also of the impurities present in the electrolytic solution.

For extremely fast switching of the brush, a potentiostat (301) that can generate AC electrochemical signals may be used where the direction of current and electron flow periodically switches back and forth at regular intervals or cycles. In this manner fast electrochemical impulses can still generate temporary pH change at the surface with rapid release of intact biomolecules bound to the working electrode.

A continuously varied reductive potential, increased pH, applied may be varied between e.g. 0 V to −1.5 V. A continuously varied oxidative potential, decrease the pH, may be varied between e.g. 0 V to +1.5 V. The specific potential used depends on the polyelectrolytic coating, the analyte, the electrodes used etc.

The average power consumption while the electrode is in operation corresponds to 0.45 mW/cm³. The power consumption required to switch the brush is low since the electrochemical signal only produces a micrometer to nanometer scale pH gradient on the surface of the electrode. The device only consumes power during the elution step of the purification. The electricity required to operate the device should be compared to the electronic equipment required by a chromatography system to manage different chemicals and liquids, as well as the cost of producing different elution buffers and the associate handling of the excess waste. By using rapid cyclic voltammetry scans the average power consumption could be substantially reduced since a shorter time is spent at the voltage with peak current, while maintaining efficient elution by electrochemical signals.

The method may further comprise a step of cleaning the device after the elution step, by applying a potential difference between the working electrode and counter electrode, where the potential difference is higher than the potential difference used during the elution. This step is analogous to a final column wash, or a regenerative wash of a chromatography column. A final wash step ensures that the column could be re-used by injecting a much stronger buffer or chemical which strips the chromatography media from any remaining analytes. Similarly an electrochemical cleaning step that exceeds the window that would otherwise be used for safe elution of analytes with a strong signal to regenerate the electrode surface for next purification. For example, applying −1.5 V to clean the surface from eventual unbound biomolecules while the safe and effective window for non-invasive separation has determined to be between −0.75 V to −1.2 V.

Alternatively, cleaning may be performed by flowing an alkaline solution e.g. 0.5 M NaOH or some other high pH solution, a high salt concentration solution, or a surfactant solution through the device to remove any unbound biomolecules that may be left on the working electrode or to any of the other internal surfaces of the device. The advantage of electrochemical cleaning would be low use of strong alkaline solutions like NaOH which is a health and environment hazard.

The device may reduce water use for purification by 52% compared to conventional pH triggered elution of analytes used in chromatography. The device may reduce time required for purification with 33% compared to conventional chromatography.

The device may reduce use of chemicals by 57% compared to conventional chromatography where chemicals are used to achieve elution.

### EXPERIMENTAL

Below follows a non-limiting description of how to produce and use the device in different applications.

### Materials

All chemicals and proteins used were purchased from Sigma-Aldrich unless stated otherwise. H₂O₂ (30%) and NH₄OH (28-30%) were from ACROS, while H₂SO₄ (98%) and ethanol (99.5%) were from SOLVECO. Water was ASTM research grade Type 1 ultrafiltered water (milli-Q-water). Chemicals used for the synthesis of diazonium salt 1 were 4-aminophenethyl alcohol, tetrafluoroboric acid (48% solution in water), acetonitrile, tert-butyl nitrate, and diethyl ether. For attaching diazonium salt to gold, L-ascorbic acid was used in water. When converting the diazonium monolayer into a polymerization initiator layer, dichloromethane, triethylamine, and α-bromoisobutyryl bromide were used. The chemicals employed in polymerization were tert-butyl acrylate, tert-butyl methacrylate, dimethylsulfoxide, dichloromethane, methane sulfonic acid, N,N,N′,N″-pentamethyldiethylenetriamine (PMDTA), CuBr₂ and L-ascorbic acid. For post modification of brushes after synthesis 1-ethyl-3-8 (3-dimethylaminopropyl) carbodiimide hydrochloride (EDC), sulfo-N-hydroxysuccinimide (NHS), N,N-bis(carboxymethyl)-L-lysine hydrate (NTA), Copper sulfate (CuSO₄), Nickel sulfate (NiSO₄) hexahydrate and protein A were used. Buffers used in this work were based on phosphate buffered saline (PBS) tablets (0.01 M phosphate, 0.13 M NaCl, pH 7.4), disodium hydrogen phosphate and NaCl, or tris(hydroxymethyl)aminomethane (TRIS) titrated to a specific pH with HCl (1 M aqueous solution) or NaOH (1 M aqueous solution). Imidazole was used to elute proteins from NTA-Me²⁺ functionalized polymer brushes.

The proteins used in this study were avidin (AVI, ThermoFisher), bovine serum albumin (BSA), lysozyme (LYS), Protein A, Lactoferrin (LAC), purified IgG from human serum, or monoclonal antibodies from CHO culture. Supernatant containing adeno-associated virus (AAV) from HEK295 culture, Clarified cell culture harvest that contains CHO supernatant containing monoclonal antibodies. Human serum (from human male AB plasma) was filtered through a 40 μm hydrophilic filter and diluted ten times in PBS prior to use.

The lipids phosphatidylcholine and dipalmitoylphosphatidylcholine, and 1,2-distearoyl-sn-glycero-3-phosphoethanolamine-N-[amino (polyethylene glycol)-2000] (ammonium salt) used to prepare liposomes, were obtained from Avanti Polar Lipids.

Quartz crystal microbalance sensors coated with stainless-steel and gold were purchased from Biolin Scientific and QuartzPro respectively. Stainless steel metal meshes with micrometer sized apertures were purchased from Anping Tianhao Wire Mesh Products Co., LTD.

### Methods

### Diazonium Salt Synthesis

The synthesis of diazonium salt involved a modified literature procedure (S. Gam-Derouich et al., Aryl diazonium salt surface chemistry and ATRP for the preparation of molecularly imprinted polymer grafts on gold substrates. Surface and Interface Analysis 42, 1050-1056 (2010)). Under an inert atmosphere, 4-aminophenethyl alcohol (2.94 g, 20 mmol) and tetrafluoroboric acid (9.94 g, 113 mmol) were dissolved in acetonitrile (20 mL). In a separate flask, tert-butyl nitrate (2.269 g, 22 mmol) was dissolved in acetonitrile (12 mL). Both solutions were degassed and cooled to −20° C. alongside 200 mL of diethyl ether.

After 20 min the solutions were warmed to 0° C., before the tert-butyl nitrate solution was added to the 4-aminophenethyl alcohol solution dropwise with stirring. The reaction was then stirred for a further 1 h. The reaction was terminated by dropwise addition of the dark yellow solution to rapidly stirring diethyl ether (200 mL). After additional stirring for 1 h the supernatant was decanted off. The brown colored precipitate was dried and 3.69 g of impure diazonium salt was obtained.

To verify the product, ¹H NMR spectra were recorded at ambient temperature on a Varian 400 MHZ NMR spectrometer. Spectra were analysed relative to external TMS and were referenced to the most downfield residual solvent resonance (CDCl₃: δH 7.26 ppm). ¹H NMR resonances of the diazonium salt matched those previously reported (S. Gam-Derouich et al., Aryl diazonium salt surface chemistry and ATRP for the preparation of molecularly imprinted polymer grafts on gold substrates. Surface and Interface Analysis 42, 1050-1056 (2010)) and analysis revealed a purity of 80%.

### Surface Cleaning

Prior to surface functionalization, QCM sensor crystals (standard Au, purchased from Biolin Scientific), and porous stainless steel foams and meshes were cleaned by washing the meshes in a mixture of hydrogen peroxide and ammonia water (H₂O:H₂O₂:NH₄OH 5:1:1 v/v at 75° C. for 20 min), followed by rinsing in milli-Q, sonication in ethanol and drying with N₂.

### Additional Metal Layer Deposition

A 50 nm gold layer was deposited on stainless-steel metal meshes to produce gold electrodes with micrometer apertures by electron-beam physical vapour deposition (Lesker PVD 225) of gold. Prior to deposition the meshes were washed with isopropanol and dried with N₂.

### Surface Activation

Gold surfaces, QCM sensors, and stainless steel meshes and foams were placed in a glass jar with a septum seal containing diazonium salt (0.301 g, 1.28 mmol) and the jar was purged with N₂. In a separate flask, ascorbic acid (0.028 g, 0.16 mmol) was dissolved in water (40 mL) and the solution was degassed for 1 h. Then, the ascorbic acid solution was transferred into the sealed glass jar causing dissolution of the diazonium salt. The gold surfaces were stirred in the solution for 1 h by use of a platform shaker (nitrogen bubbles that appear on the surface after 15 min indicate successful diazonium salt monolayer formation), after which they were thoroughly rinsed in water then ethanol, and dried.

To convert the diazonium monolayer (the monolayer is illustrated in FIG. 5b 501 into a polymerization initiator layer, the gold surfaces were exposed to α-bromoisobutyryl bromide (0.222 mL, 1.80 mmol) and triethylamine (0.302 mL, 2.17 mmol) in dichloromethane (20 mL) for 10 minutes, after which surfaces were rinsed in ethanol and dried under N₂.

### Surface-Initiated Polymerization

SI-ATRP (surface initiated activator-regenerated atom transfer radical polymerization) was used to prepare poly(acrylic acid) (PAA) polymer brushes, i.e. the polyelectrolytic coating, in a manner similar to published procedures (G. Ferrand-Drake del Castillo, G. Emilsson, A. Dahlin, Quantitative analysis of thickness and pH actuation of weak polyelectrolyte brushes. J Phys Chem C 122, 27516-27527 (2018)).

Inhibitor was removed from the monomer tert-butyl acrylate (TBA) using an alumina column, after which it were stored at −20° C., then warmed to room temperature immediately before use. Reactions were carried out using standard Schlenk line techniques under an inert atmosphere of N₂. CuBr₂ (0.006 g, 0.03 mmol), and pentamethyldiethylenetriamine (PMDTA), (0.056 mL, 0.276 mmol) were dissolved in dimethyl sulfoxide (20 mL) and, alongside a separate flask of tert-butyl acrylate (20 mL, 0.1378 mol), was deoxygenated via vigorous bubbling of N₂ for 30 min.

The reaction solution and monomer were then transferred via cannula into a screw-top jar (with rubber septa lid) containing initiator-prepared gold surfaces. The reaction was initiated by the addition of ascorbic acid (0.049 g, 0.276 mmol). The final concentrations of each component in the reaction medium were: [monomer]=3.4 M, [CuBr₂]=1.1 mM, [PMDTA]=11.0 mM, and [ascorbic acid]=11.0 mM. The reaction was placed under magnetic stirring. Reactions were quenched by immersing the samples in pure ethanol. Poly(tert-butyl acrylate) (PTBA) brushes were then converted to PAA by exposure to 0.2 mM methane sulfonic acid in dichloromethane (10 mL) for 15 min, followed by rinsing in dichloromethane and ethanol.

### Post Modification of Polymer Brushes

Polymer brushes were modified after polymerization by conversion of the carboxylic acids of PAA to alter the protein binding properties either by attaching at metal ion complex NTA-Me²⁺ or by immobilization of Protein A. EDC/NHS coupling technique was employed where 50 mM EDC and 50 mM NHS was dissolved in water. The electrode surface was exposed for 30 minutes to this solution followed by rinsing in water. For metal ion complexation, an NTA solution was prepared 100 mM and set to pH 10, the electrode was exposed to this solution for one hour followed by rinsing in water. A divalent metal ion was attached by exposure to a 100 mM solution of CuSO₄ or NiSO₄ for 30 minutes followed by rinsing in water. For Protein A immobilization the electrode surface was treated with the same EDC/NHS activation, but instead of exposure to NTA and metal ion solutions the electrode was immersed in a solution of Protein A 0.3 g/L at pH 7.4 for one hour followed by rinsing in water.

### Preparation of Liposomes

Liposomes containing predominantly DPPC lipids mixed with lipids with headgroups containing either net cationic functional groups (5%) or PEG 2000 kDa polymers (5%) were prepared using an Avanti Mini Extruder kit. Nanoparticle tracking microscopy was used to verify liposome size, the distribution of liposome sizes of the sample, and to compare the size and distribution before and after capture and release to the working electrode.

Protein Immobilization to and Release from Polymer Brush Coated Electrodes

Immobilization of proteins to polymer brushes on an electrode surface was conducted in one of three ways:
1. Immobilization of any protein or collection of proteins to a PAA functionalized electrodes. The buffer solution (electrolytic solution) was composed of phosphate (5 mM) and sodium chloride (75 mM) set to pH 5.0. First the electrode was equilibrated in the buffer solution, following equilibration the sample was exposed to the protein solution (5 g/L). Subsequently the electrode was rinsed in the same buffer solution to remove loosely bound proteins. Elution was performed by (i) application of negative (reductive) potentials (−0.3 V to −1.2 V) where the magnitude of the potential and duration of the potential determined the rate and quantity of release from the electrode surface. (ii) changing the solution pH to a basic pH value ranging between pH 6 to pH 11.5 such that the brush becomes sufficiently charged to start repelling the proteins bound to the polymer brush, or repelling a fraction of the bound proteins to the brush.
2. Immobilization of recombinant proteins with a His-tag to NTA-Me²⁺ functionalized electrodes. The electrode was equilibrated in the background buffer solution with composition TRIS (50 mM) and NaCl (250 mM), followed by injection of polyhistidine-tagged proteins (5 g/L). The electrode was rinsed in the background buffer solution to remove loosely bound proteins. Elution of His-tagged proteins was accomplished by (i) reductive (negative) potentials reducing the divalent metal ions of the NTA-Me²⁺ ligand breaking the metal-ion coordination bond with the His-tagged protein (ii) An additional option for electrodes with a gold surface: Exposure to a hydroquinone solution (5 mM) and a positive (oxidative) potential (+0.3 to +0.6 V). (iii) the final method was to expose the surface to a 250 mM imidazole solution.
3. Immobilization of antibodies to Protein A functionalized gold electrodes. The background buffer was composed of neutral buffer pH 7.4 (electrolytical solution). After equilibration of the electrode surface in the background buffer it was exposed to a solution of antibodies (0.25 g/L), following this the electrode was rinsed in background buffer to remove loosely bound or unbound antibodies. Elution was achieved by (i) exposure to a 5 mM hydroquinone buffer solution combined with an oxidative (positive) potential (+0.3 to +0.6 V) (ii) changing the solution pH to an acidic value between pH 2-3, resulting in release between the protein A and antibody ligand interaction.

### Electrochemical QCMD Measurements

Sensor crystals coated with gold or stainless steel (316L) were used and measurements were performed using a Q-Sense E4 (Biolin Scientific). All data shown corresponds to the first or third overtone. A flow cell with an electrochemical module (QEM 401) was used to perform in-situ electrochemical experiments. A Gamry Interface 1010E potentiostat (Gamry Instruments) was connected to the electrochemical cell. For every experiment the internal resistance of the circuit was measured (Get Ru) and the open circuit potential was measured to verify an acceptable reference electrode performance and correctly connected circuit. The reference electrode used was a World Precision Instrument low leakage “Dri-ref” electrode. The scan rate in CV experiments was 100 mV/s.

### Reference Electrode Preparation

The reference electrode of the device was prepared by depositing chloride ions onto a bare silver wire electrochemically by applying a +1.0 V for 5 min in concentrated HCl diluted by 10×. To make the reference electrode part the silver wire was threaded through a 3D-printed nut with a hollow opening on one end, a small hole with a diameter that closely matches the silver wire, and a threading that allowed it to be tightly screwed onto the device. The tip of the silver wire coated with AgCl, intended to be interfaced with the electrolytic solution, was positioned inside the hollow space within the nut. The other uncoated end of the wire was positioned such that it protruded out from the top of the nut allowing it to be connected to the potentiostat. The silver wire and the 3D-printed part was glued together such that the wire was fixed in its position and that the opening for the reference electrode was waterproof once the reference electrode nut was connected.

### 3D-Printing of Device

Three-dimensional models of the device, embodiment shown in FIGS. 1a and 1b, were designed in CAD software. The prototype was sliced in PrusaSlicer (Prusa3D) and printed on a MK3S 3D-printer (Prusa3D) using poly(ethylene terephthalate glycol) (PETG) or polypropylene (PP) filament using print settings (0.1 mm layer height, 5-6 wall layers, 5% extrusion multiplier, 100% fill density) to make the device resistant to leakage.

### Assembly of Device

The device was assembled as shown in FIG. 1b, by threading the centre body piece 115 onto the inlet body piece 114. The reference electrode 103 was connected to the inlet body piece 114. The counter electrode 102 was placed on the internal surface of the inlet body piece 114, after which a spacer element 107 and an o-ring were positioned above the counter electrode 102 such that the o-ring has full contact with the inlet body piece. The working electrode placed lying on the spacer element disc and the outlet body piece was connected by threading it into the centre piece. Connector pins for the working and counter electrode comprising metal screws in stainless steel were threaded onto respective inlet ports until they made contact with respective electrode.

### Method for Use of Device on Commercial Chromatography Systems

The device was connected using M6 connector threading to a commercial chromatography system ÄKTA Explorer (Cytiva). The protein separation was monitored using inline UV light detectors and evaluated by analysis of eluted sample aliquots.

Each experiment started with connecting the inlet of the device to the chromatography system and flushing the prototype with water for 10 column volumes (1 CV=1 mL). Following this the device output was connected to the chromatography system, the system was equilibrated in PBS pH 5 by washing the system and rinsing through the device connected in-line.

A protein sample consisting of either one protein (e.g. BSA), a mixture of proteins, or a serum sample, (1 mL) was injected into a sample port into a sample load line (3 mL total capacity). The experiment was started where the UV absorbance was measured with an in-line UV monitor with fixed wavelength detection at 215 nm and 280 nm. First, the sample was loaded onto the column at a flow rate of 0.1 mL/min for 30 min. After loading the flow rate was increased to 0.5 mL/min. When the signal had stabilized, electrochemical elution was performed to elute proteins from the device. Alternatively, bulk solution pH increase was used to elute proteins from the device. Prior to using electrochemical elution the open circuit potential (OCP), solution resistance (Get Ru) and a cyclic voltammetry sweep cycle (from 0 V to −0.5 V at 100 mV/s) was applied to check that the three-electrode system was properly configured.

### EXAMPLES

The examples below are provided for illustrative purposes only and should not be construed as limiting.

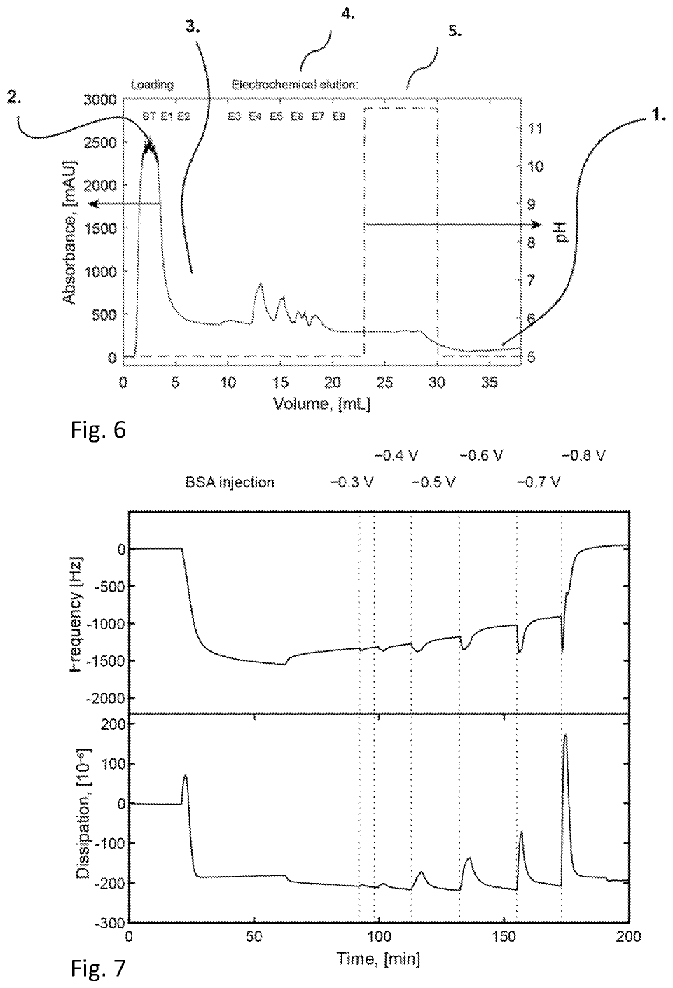

### Example 1: Use of the Device for Electrochemical Biomolecule Separation with a PAA Functionalized Electrode Surface, Characterized by the Following 5 Steps. Each Step 1-5 is Indicated in the Chromatogram in FIG. 6

### 0. Testing the Electrochemical Signal

A test of the electrochemical configuration of the system is performed, where the following experiments are performed when the device contains a buffer solution: open circuit potential (OCP), solution resistance measurement (Get Ru), and cyclic voltammetry scans, are performed to ensure efficient electrochemical signals can be established between the electrodes of the device. A useful (OCP) signal is characterized by being stable and falls within +0.5V, the solution resistance is characterized by being low and within acceptable limits set by the potentiostat manufacturer, A cyclic voltammetry scan is characterized by having a peak current of 1-5 mA/cm² of electrode geometrical surface area at −0.5 V.

### 1. Rinse and Equilibration

The device is connected to a liquid management system comprising a pump, pump valves, buffer solutions, in-line monitoring sensors (UV-optical, pH, conductivity). The background buffer (electrolytic solution) is used to equilibrate the system at the selected pH and salt concentration where separation is to be conducted. In this example the composition of the electrolytic solution is characterized by being pH 5.0, phosphate buffer concentration of 5 mM, and a total ionic strength salt concentration of 75 mM. Equilibration is monitored using the in-line sensors of the chromatography system.

### 2. Sample Binding

Sample containing an analyte and other impurity components from which is injected through the inlet by the liquid management system. Onset of breakthrough indicates that at least a part of the sample solution has passed through the device, if analyte flows through the device either the rate of binding of biomolecules to the electrode surface of the device is not sufficient to bind all sample analyte, or all the binding sites on the electrode surface has been occupied with a binding analyte. (Breakthrough here is defined as the point during sample binding when biomolecules are detected by the in-line sensor monitors positioned after device outlet)

### 3. Rinse

After binding to working electrode the device is rinsed with buffer until unbound biomolecules have been evacuated from the interior volume of the device, characterized by a decrease and stabilization of the in-line UV signal.

### 4. Elution

Elution of bound biomolecules to the working electrode can be achieved by an electrochemical signal that alters the surface pH and thereby the intermolecular interactions between the polymer brush on the working electrode and the bound biomolecules. Elution can also be achieved by changing the entire solution pH.
Electrochemical elution can be performed by:

A1. Application of a constant electrochemical potential. When a constant potential is applied a pH gradient is established. A2. By application of a variable electrochemical potential. A variable electrochemical potential will establish a variable pH gradient where in addition to the above mentioned effects the rate of potential change will affect the extension of the pH gradient and result in a temporal variation in the change of the surface pH. The extent of the electrochemical pH gradient is determined by: (i) the buffer capacity of the solution which counteracts the electrochemical reaction that alters the surface pH, (ii) The magnitude of the electrochemical potential which determines the rate of the electrochemical reaction and thereby the rate of pH change on the surface. (iii) The flow rate through the device and design features that affect mass-transfer to and from the electrode surface (iv) The duration of the electrochemical signal that removes transient elements in establishing the pH gradient.

By adjusting these factors, buffer capacity, potential window, flow speed, duration of the signal, a specific local pH value confined to the surface of the electrode is obtained, whereby a release of a specific analyte bound to the surface can be triggered. By tuning of the potential separation of a specific biomolecule that releases at a certain pH value can be achieved where elution of a pure sample occurs, which can be collected in separate liquid aliquot samples by a fraction collector of the liquid management system.

Elution by changing the entire pH solution can be performed by changing the solution pH by pumping a buffer with a different pH through the device. Analogous to changing the surface pH, changing the solution pH will result in elution of bound biomolecules.

### 5. Cleaning

Cleaning after elution may be performed by
I. Flowing an alkaline solution e.g. 0.5 M NaOH or some other high pH solution, a high salt concentration solution, or a surfactant solution through the device to remove any unbound biomolecules that may be left on the working electrode or to any of the other internal surfaces of the device.
II. Electrochemical cleaning of the working electrode to remove any eventual unbound biomolecules bound to the working electrode, by application of a slightly higher potential than required to achieve a high temporary surface pH resulting in stripping, regeneration and complete cleaning of the surface of the working electrode without disassembly or flow through of a cleaning solution e.g. 0.5 M NaOH.
   Steps 1-5 are repeated when a new sample of analytes/biomolecules is injected into the inlet process stream.
   Clean-in-place with an alkaline solution like NaOH, or stripping with an extra strong buffer solution is in principle optional with the device described here-in. Complete cleaning of the electrode is achievable by optimization of the electrochemical signal and exposure of the entire electroactive surface area of the electrode. Therefore step 5 could be completely eliminated for the device described herein.
   The device has a potential to enable substantial productivity gains in the purification by saving volume of water, time, and chemicals required. Table 1 summarizes the conventional steps used in chromatography followed by the corresponding minimum steps required to achieve purification for the device described herein. A theoretical column volume of 1 L is used to calculate the quantity of chemicals, a buffer composition of 0.02 M and 0.15 M salt concentration was used, clean-in-place (CIP) step was calculated to require 0.5 M. Neutralization of highly acidic pH buffer used for elution is included in the calculation for conventional chromatography, however it is not required for electrochemical elution.

TABLE 1
Comparison of Column volumes, time and chemical quantity
in moles used for purification of an analyte for conventional
chromatography and device described herein.

| | | | | Current | | |
|---|---|---|---|---|---|---|
| | Conventional chromatography | | | device | Time, | |
| Step | CV | Time, min | n, mol | CV | min | n, mol |
| Equilibration | 3 | 10 | 0.51 | 3 | 10 | 0.51 |
| (1) | | | | | | |
| Sample | 7 | 30 | | 7 | 30 | |
| application (2) | | | | | | |
| Wash 1 (3) | 5 | 10 | 0.85 | 1 | 10 | 0.17 |
| Wash 2 | 1 | 10 | 0.52 | — | — | 0.52 |
| Elution (4) | 3 | 20 | 0.15 | 3 | 20 | 0 |
| Strip (5) | 2 | 10 | 0.2 | — | — | 0.1 |
| CIP (5) | 2 | 20 | 1 | — | — | 0.5 |
| Re-eq (1) | 5 | 10 | 0.85 | 1 | 10 | 0 |
| Neutralization | 3 | — | 0.15 | — | — | |
| Total | 31 | 120 | 4.23 | 15 | 80 | 1.80 |

Table 2 shows a summary of the gains in productivity that use of the device could result in in terms of water use, time for purification and chemicals use required for purification. The device could lower water use by 52%, time by 33% and chemical use by 57%.

TABLE 2
Comparison of total water use, time consumption and
chemical use for chromatography compared to device
and the corresponding reduction in materials use that
may be achieved by using the device described herein.

| Comparison | Chromatography | Nyctea | Reduction % |
|---|---|---|---|
| Water volume, L | 31 | 15 | 52% |
| Time, min | 120 | 80 | 33% |
| Chemical use, mol | 4.23 | 1.80 | 57% |

### Example 2: Capture and Release of Bovine Serum Albumin (BSA) from PAA Functionalized Stainless Steel QCM Sensors

Quartz crystal microbalance with dissipation monitoring (QCMD) was used to sense in real time how a surface functionalized with a PAA brush prepared using SI-ATRP and anchored to the stainless steel using diazonium salt chemistry, responds to exposure to protein solutions, and to electrochemical signals.

FIG. 7 shows a QCM sensorgram with frequency and dissipation signals monitored during a protein capture and release experiment. BSA (0.3 g/L) is captured at pH 5.0 in large quantities as verified by a shift in frequency that exceeds 1000 Hz. This corresponds to multilayers or BSA immobilized within a predominantly neutral brush but where the degree of hydration is large enough to accommodate multiple layers of proteins.

Following immobilization and a rinse step, electrochemical signals are used to reversibly charge the PAA polymer brush giving rise to tunable controlled release of proteins from the surface. Applying potentials with increasingly higher magnitude indicates that the release of proteins due to degree of charging is influenced by the magnitude of the potential, and the duration of the potential.

FIG. 7 shows that PAA functionalized of stainless steel surfaces have the electrocatalytical ability to perform oxygen reduction reactions at a rate that produces substantial basic pH gradients resulting in reversible switching of the PAA brush that enables tunable biomolecule release. We know from previous work that noble metals (WO2021/107836) are sufficiently good electrocatalyst for producing these interface electrochemical pH gradients. FIG. 7 highlights that there is industrial applicability for large scale protein separation using electrochemistry since it is possible to use abundant non-noble metals like stainless steel as the solid support electrode material of the present device.

### Example 3: Capture and Release of BSA from Porous Stainless Steel Mesh Electrodes Functionalized with Poly(Acrylic Acid) PAA Brushes when Connecting the Device to a Commercial Chromatography System

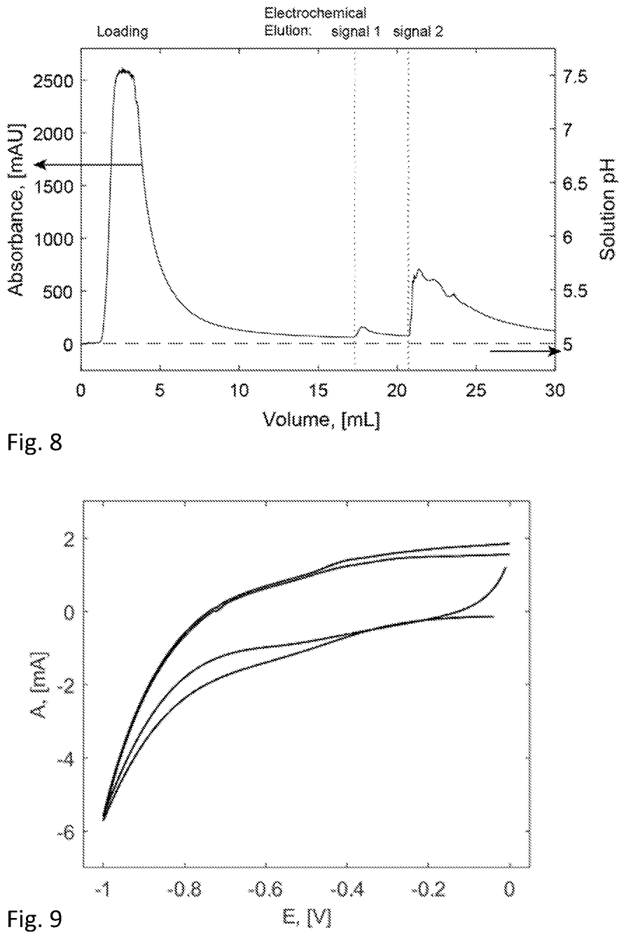

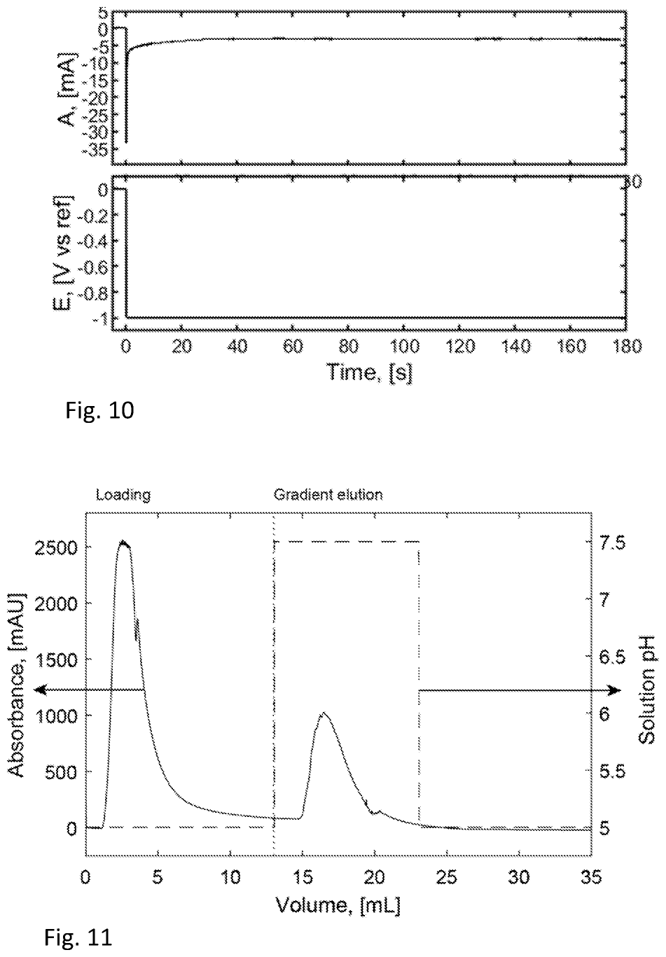

FIG. 8 shows loading of BSA (5 g/L) to porous stainless steel mesh electrodes. After loading and rinsing of unbound proteins electrochemical elution was achieved in two steps. First cyclic voltammetry scans were applied (0 V to −1 V), see FIG. 9, resulting in a small elution peak. In the second elution signal a constant negative potential −1 V was applied which resulted in a larger peak, see the corresponding chronoamperometry signals obtained from the potentiostat in FIG. 10. Both signals produce a substantial electrochemical pH gradient that switches the brush with subsequent release of proteins detected in the outflow from the device. The magnitude of the electronic signal in terms of voltage is low, where protein release is detected already at −0.5 V. The peak current density for a variable potential (signal 1) is 6 mA/cm² electrode surface, while the peak current density for a constant potential set to −1.0 V is around 32 mA/cm². Both methods for switching the brush give rise to very brief spikes in current density, indicating that very small power output is required to establish a pH gradient electrochemically. A low current density over time is expected since the pH gradient is rapidly established once the signal is turned on, and only extends around ˜1 μm away from the surface of the electrode. When the electrochemical pH gradient is established, the electro catalytical reaction will be rate limited by the mass transfer of new reactants, which contributes to a lower the electron transfer across the electrode interface and a low power output and average current magnitudes below 1 mA/cm² over extended periods of operation.

Upon application of a negative electrochemical signal a temporary pH gradient is established on the surface of the stainless steel mesh surface. This induces charging of the PAA polymer brush within porous structure resulting in breaking of the hydrogen bonds with BSA molecules and electrostatic repulsion between the brush and the protein. The applied signal magnitude determines the degree of elution allowing for tunable electrochemical release from the device with clear analogy to the results shown in QCMD and FIG. 7. However, contrary to the QCMD experiment where a flat surface of stainless steel is used to capture and release proteins, in the chromatogram of FIG. 8 the protein sample passes through an electrode with micrometre aperture. When the proteins sample flows through the device the quantity of captured and eluted proteins becomes substantially higher than with QCMD sensors, since the effective surface area of the device is much larger. By integration of the binding peak and the elution peaks the loading capacity of the device was estimated. The binding capacity from electrochemical elution was determined to be 29 mg BSA/cm³ volume of electrode mesh. There is a clear improvement in the volumetric binding capacity for a porous electrode mesh compared to a flat surface like the QCMD sensor which can store ˜1 μg/cm² making the device useful in commercial applications where a high binding capacity is a requirement.

Changing the entire solution pH, through gradient elution, represents an alternative method of eluting BSA from the stainless steel mesh, as shown in FIG. 11. When the solution pH reaches a sufficiently high value PAA becomes charged, resulting in electrostatic repulsion between BSA and PAA and BSA elutes from the device resulting in a peak in the chromatogram. The corresponding binding capacity when eluting by increasing the solution pH elution was 31 mg BSA/cm³. This shows that the electrode mesh is capable of efficient release by electrochemical signals, matching the eluted quantity of protein when performing elution by changing the solution pH. Consequently elution results are the same regardless if electrochemical of pH solution elution is performed.

Larger binding capacity that matches or even exceeds values obtained for microporous resin-based chromatography, or membrane-based chromatography is possible by engineering the electrode material to have large surface area, combined with using polymer brushes which in addition boosts the binding capacity per surface area.

Complete evacuation of proteins was achieved by electrochemistry meaning that is it possible to re-set the device by releasing all proteins bound making it possible to re-use the device and perform another protein loading cycle.

### Example 4: Capture and Release of a Mixture of Proteins, BSA, Lactoferrin (LAC) and Lysozyme (LYS) from Porous Stainless Steel Mesh Electrodes Functionalized with Poly(Acrylic Acid) PAA

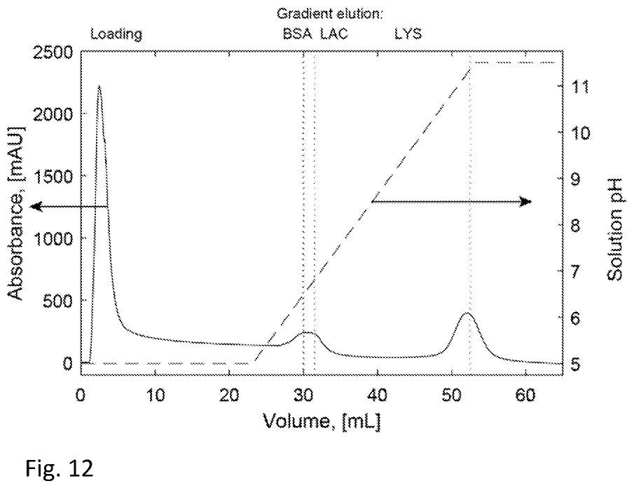

Mixtures of proteins can be separated into fractions with the device. FIG. 12 serves a proof of this concept that a mixture of proteins, in this case BSA, LAC and LYS, are separated by a solution pH gradient. FIG. 12 shows a chromatogram where a mixture of said three proteins are bound to the electrode within the device at pH 5.

Followed by loading and saturation of the proteins on the working electrode, the device was rinsed with pH 5 buffer solution. A pH gradient was applied where BSA elutes first since it has the lowest (pI=4.2), followed by Lactoferrin (pI˜8.7), followed by lysozyme (pI=11). This demonstrates that the pH of the solution supplied in the flow through the device produces elution. Similarly, application of a local pH gradient by electrochemical signals will then also produce separation between different proteins (not shown).

### Example 5: Separation of a Complex Biomolecule Mixture, Human Serum, into Fractions of Proteins by Capture at pH 5 and Physiological Salt Concentration, Followed by Application of Electrochemical Signals Using Porous Stainless Steel Mesh Electrodes Functionalized with Poly(Acrylic Acid) PAA

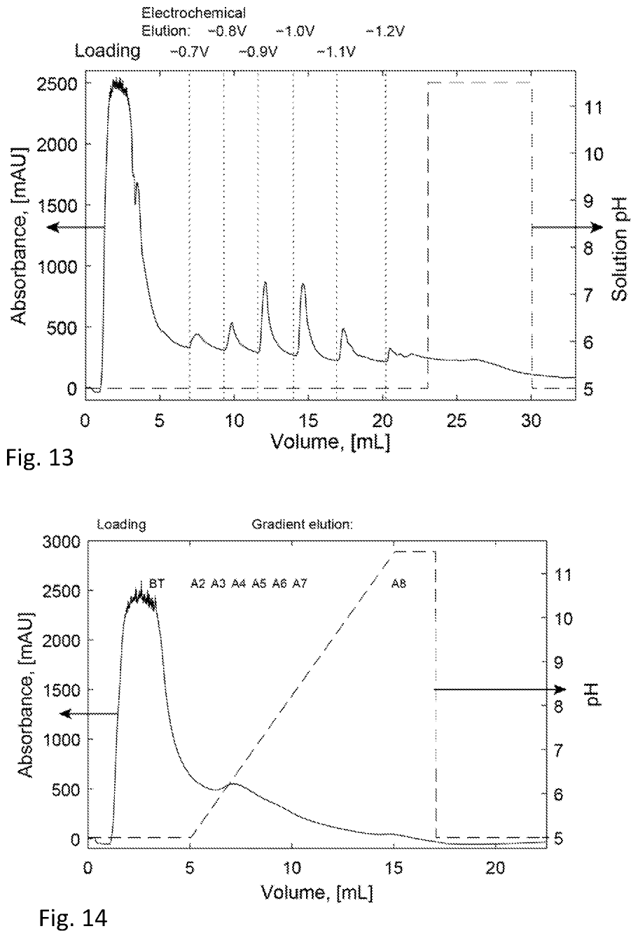

FIG. 13 shows a chromatogram where a sample of human serum is diluted 10× in PBS, the pH is set to pH 5.0. First biomolecules are captured by the device, followed by breakthrough of uncaptured proteins when the polymer coated surface of the working electrode becomes saturated with serum biomolecules. After rinsing with buffer, electrochemical signals of increasing magnitude were applied showing resulting in sharp peaks in the chromatogram confirming that the electrochemical potentials give rise to well defined sharp concentrated elution events. The duration of the elution can be controlled on-demand and in real time and the relative quantity of product that is eluted can also be tuned by adjusting the magnitude of the applied potential.

FIG. 14 shows elution without electrochemistry, by gradually increasing the solution pH to higher values than pH 5.0 within the device results in elution of proteins, the pH increases as higher pH solution is injected, which eventually leads to charging of the polyelectrolyte coating, followed by breaking of the hydrogen bonds between proteins and the polyelectrolyte brush coating of the working electrode. The separation between proteins bound can be adjusted by setting a shallow or steep gradient. A shallow gradient, slow pH increase, separates the elution peaks, but at the cost of high dilution and low final concentration of the product. A steep gradient results in poor separation but the product but with less dilution. Elution by a solution pH gradient as shown in FIG. 14 results in broad peaks with low signal magnitude because it requires shifting the pH of the entire solution within the device. Electrochemical elution as shown in FIG. 13 is characterized by sharp discrete elution peaks because it rapidly shifts the pH on the surface of the working electrode where the bound proteins are located. Sharp elution peaks have a clear advantage since it results in higher concentrations of product that is eluted. Furthermore, it removes the need of an additional substance for changing the pH to elute bound proteins.

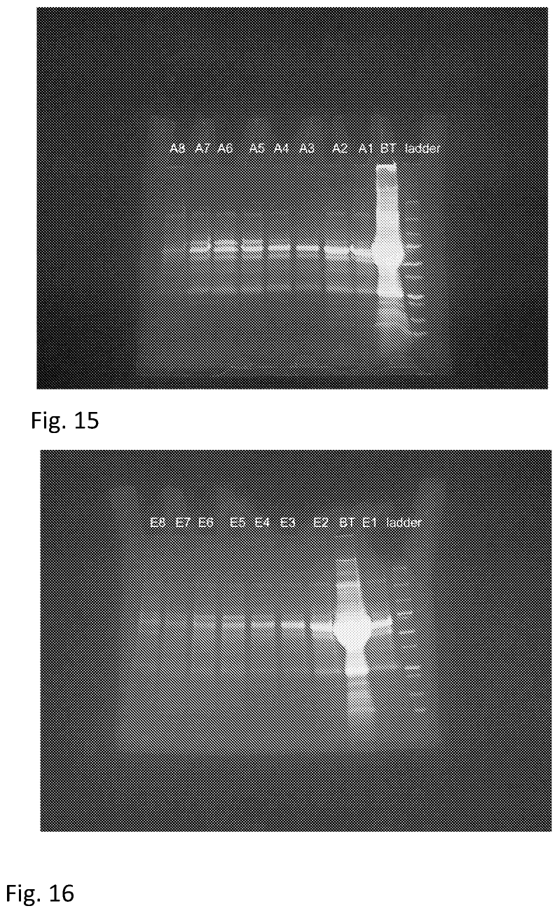

FIG. 15 and FIG. 16 are photographs of SDS-PAGE gels used to analyse the collected fractions obtained from chromatography experiments (chromatograms shown in FIGS. 6 and 14) with solution pH and electrochemical elution, respectively. The analysis of the SDS-PAGE gels reveals that protein are eluted by both methods, that proteins of certain sizes are enriched in the sample fractions (E5-E7 and A5-A7) collected while applying different magnitudes of electrochemical potentials and while increasing the solution pH respectively. Confirming that said device performs separation of serum proteins and biomolecules, controlled by electrochemical signals, resembling the separation obtained by gradually increasing the solution pH that flows through the device.

### Example 6: Separation of Complex Biological Fluid, Like Human Serum, into Pure Proteins by Capture at Neutral pH and Reduced Salt Concentration Followed by Electrochemical Release from Porous Stainless-Steel Electrodes Functionalised with Poly(Acrylic Acid) PAA

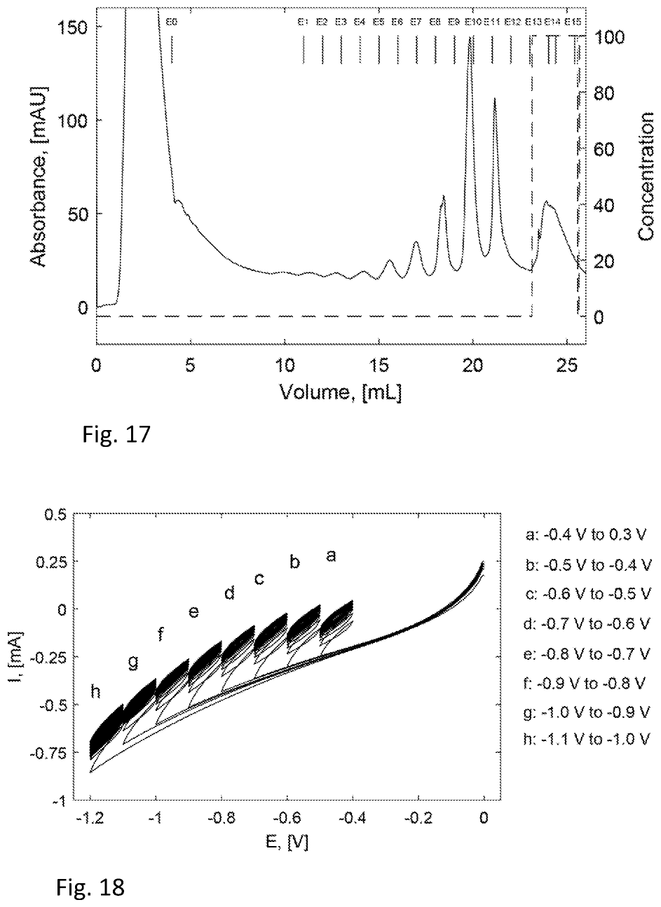

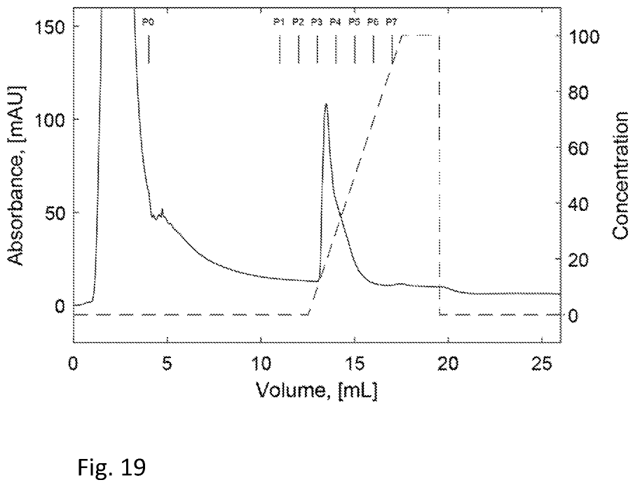

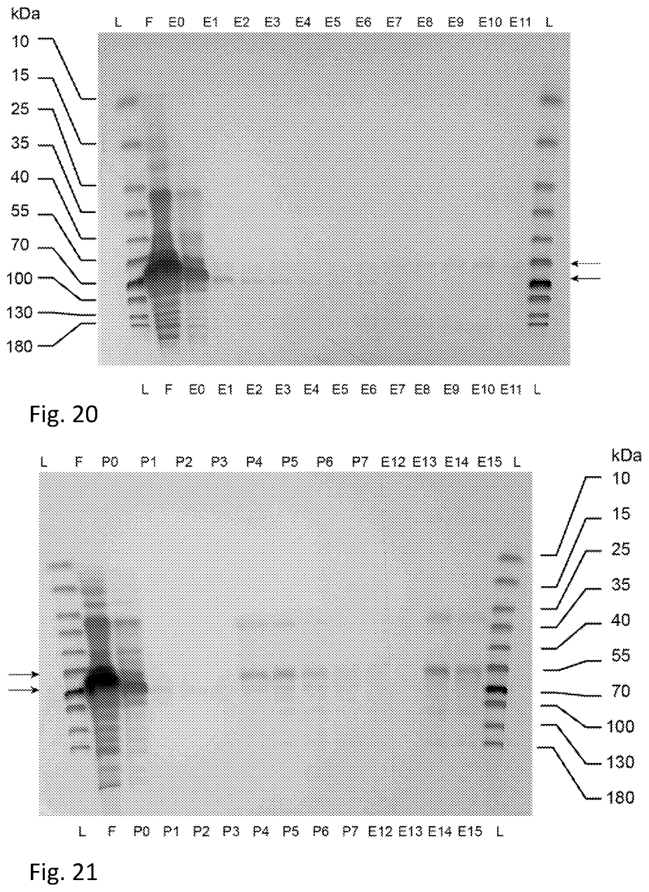

For separation of some biological solutions, it may not be possible to lower the pH of the sample to pH 5 in order to bind the sample to the electrode coating, due to instabilities of the sample towards solution pH change. An alternative method to trigger binding to the brush is to lower the salt concentration. This shifts the pKa of the polyelectrolytic coating to higher values, resulting in a protonated neutral PAA coating that binds the sample molecules at neutral pH 7.0-7.5. FIG. 17 show capture of human serum diluted with a weak 0.1×PBS buffer at pH 7.0 followed by electrochemical elution by applying a series of cyclic voltammetry scans with increasing magnitudes, shown in FIG. 18. The corresponding chromatography for solution pH release is shown in FIG. 19 where increasing in-mixing of 0.1M NaOH is used to raise the solution pH and thereby trigger release of bound serum biomolecules. In both chromatography experiments sample fractions were collected to compare by SDS-PAGE the separation achieved by electrochemistry, FIG. 20 and solution pH, FIG. 21. Sample fractions collected during electrochemical signals with peak voltage between −0.4 V and −0.8V triggered release of a protein slightly smaller than 70 kDa, whereas larger magnitude potentials between −0.9 to −1.2 V resulted in elution of a smaller protein near 55 kDa, as indicated by the arrows in FIG. 20. A corresponding separation resolution of samples collected during a gradient in solution pH elution was not produced, FIG. 21, instead both protein species are found eluted into the same samples as indicated by the arrows. A smaller protein species was detected in FIG. 21 yet it was not clearly observed in fractions by electrochemical elution FIG. 20. Comparison of the gels demonstrate how the device can produce a different separation compared to conventional ion-exchange mediated by solution pH changes. The separation is conducted at neutral pH value and only briefly exposes the sample to pH change at the nanoscale of the electrode interface, any electrochemically mediated pH change in the solution is quickly reversed as the buffer fluid leaves the device, an important feature for purification of pH sensitive biomolecules.

### Example 7: Affinity Tag Binding and Electrochemical Elution, Replacing Elution by Imidazole, of a Recombinant Protein with a Polyhistidine Tag from a QCMD Sensor Electrode Functionalized with NTA-Me²⁺ Polymer Brushes

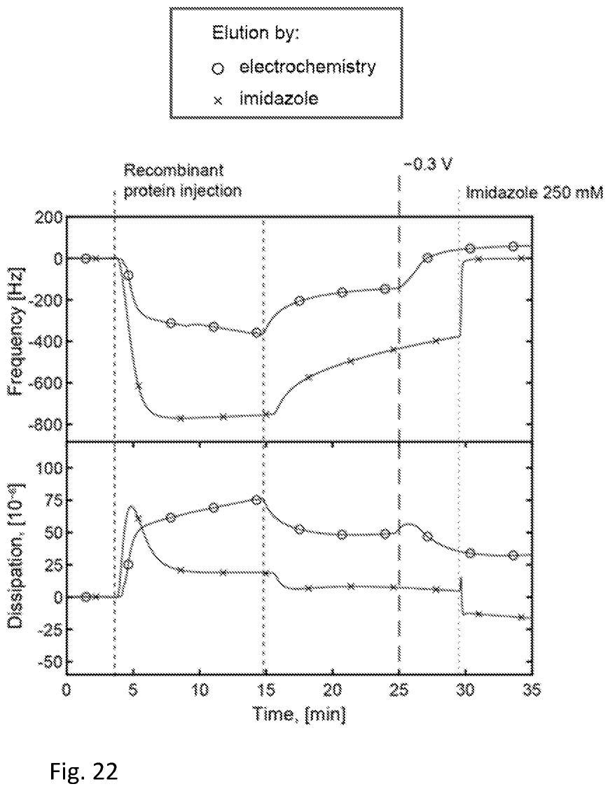

FIG. 22 shows spontaneous binding of a recombinant protein to a polymer brush with NTA-Me²⁺ functional groups, converted from a PAA brush using EDC/NHS coupling chemistry protocol. Recombinant proteins were expressed with a His-tag which will bind specifically to the NTA-Me²⁺ ligand on the brush as verified by shifts in the frequency signal by several hundreds of Hz. Traditionally elution is performed by imidazole 250 mM as shown in one of the sensorgram in FIG. 22, circles. However, release by electrochemical signals was also performed by application of a weakly reductive potential, reducing the Cu²⁺ ions causing a release of the bound recombinant proteins. This method offers an alternative way for high-capacity reversible elution of His-tagged proteins using electrochemistry without the need of adding a chemical additive to trigger elution like imidazole.

### Example 8: Affinity Tag Binding of Antibodies on a Protein a Functionalized Polymer Brush and Electrochemical Elution on a QCMD Sensor Electrode Replacing Conventional Acidic, Low pH Solution Elution

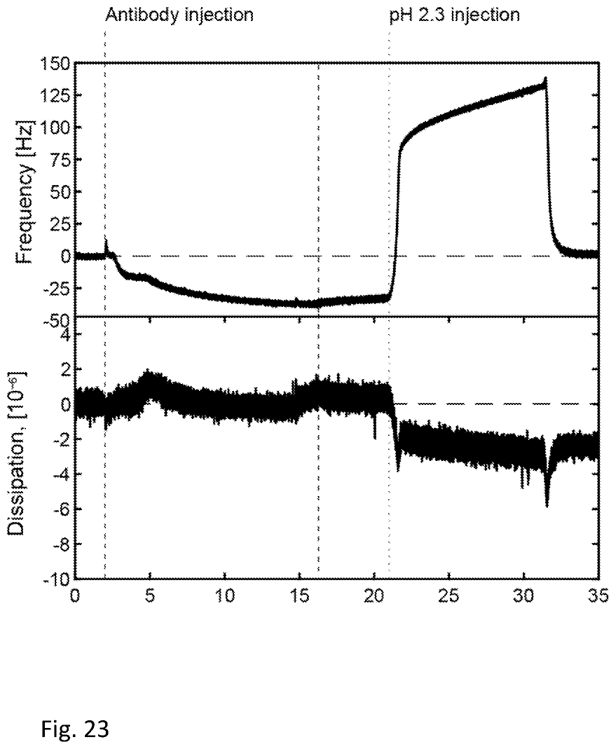

FIG. 23 shows spontaneous binding of antibodies to a protein A functionalized surface using EDC/NHS. The dissipation signal changes that occurs upon binding of antibodies to the surface are lower compared to the corresponding dissipation shift observed when binding BSA to polyacidic brushes (FIG. 7). This indicates that the brush is less hydrophilic and swollen, due to that a majority of the carboxylic acids within the brush have successfully undergone conjugation with Protein A through the EDC/NHS treatment. It is therefore reasonable to assume that the interactions that occur between the surface and the antibodies are due to the highly specific interaction that occurs between the Protein A and the antibody binding regions (in particular the Fc region of IGG), and not with carboxylic acids. Antibodies spontaneously bind to Protein A on the surface when the pH is set to 7.4. The bound antibodies remain bound when the surface is rinsed with pH 7.4 buffer, but when the surface is exposed to pH 2.3 for ten minutes the bound antibodies are released from the surface. This is expected since industrial purifications of antibodies is performed by chromatography where Protein A coated porous resin materials are used for highly specific capture, followed by low pH elution wash.

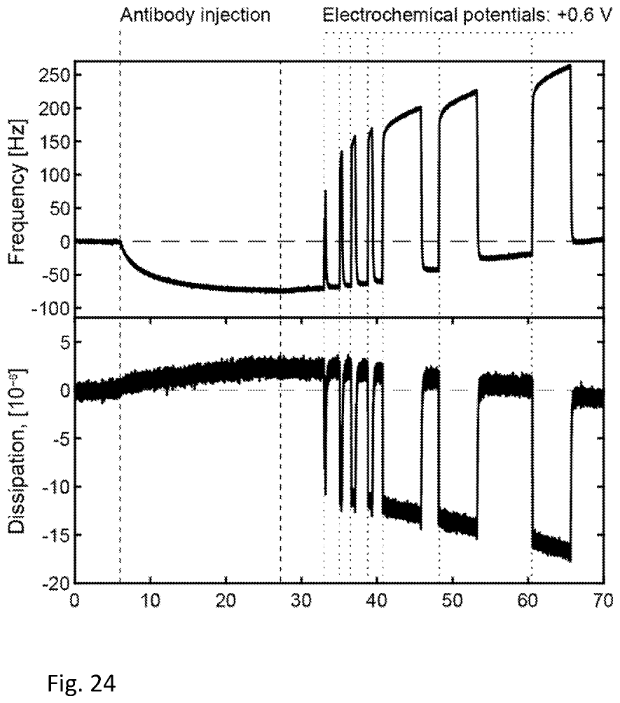

Alternatively, as is shown in FIG. 24, protein A coating can elute captured antibodies without needing an acidic solution wash. Instead, release from the coating is performed by producing a local low pH gradient when a positive electrochemical potential is applied in the presence of 5 mM hydroquinone. Hydroquinone undergoes oxidation resulting in production of protons at the interface, which lowers the pH temporarily, but sufficiently long for the Protein A-IGG ligand bond to be disrupted. When a constant potential, +0.6 V is applied for 5 minutes a significant quantity of antibodies releases from the surface. After three repetitions, full elution was achieved since the signal returned to the baseline which means that all bound antibodies have been removed from the surface. By adjustment of the electrochemical signal, the concentration of hydroquinone, or by selection of another reduction agent the magnitude of the pH gradient may be tuned to increase the speed and efficiency of the electrochemical elution.

In summary FIG. 23 and FIG. 24 show how polyacidic brushes can be used to bind large quantities of Protein A to a post-functionalized polyelectrolytic coating to capture proteins by highly specific biological ligand interactions, and in extension, how these biological interactions also can be locally tuned on the surface using electrochemical potentials for elution instead of using solutions with extremely low pH.

### Example 9: Electrochemical Purification of mAb from Clarified Cell Culture Harvest Using a Protein a Functionalized Microporous Stainless Steel Mesh Electrode, Replacing Problematic Elution by Acidic Low pH Solution

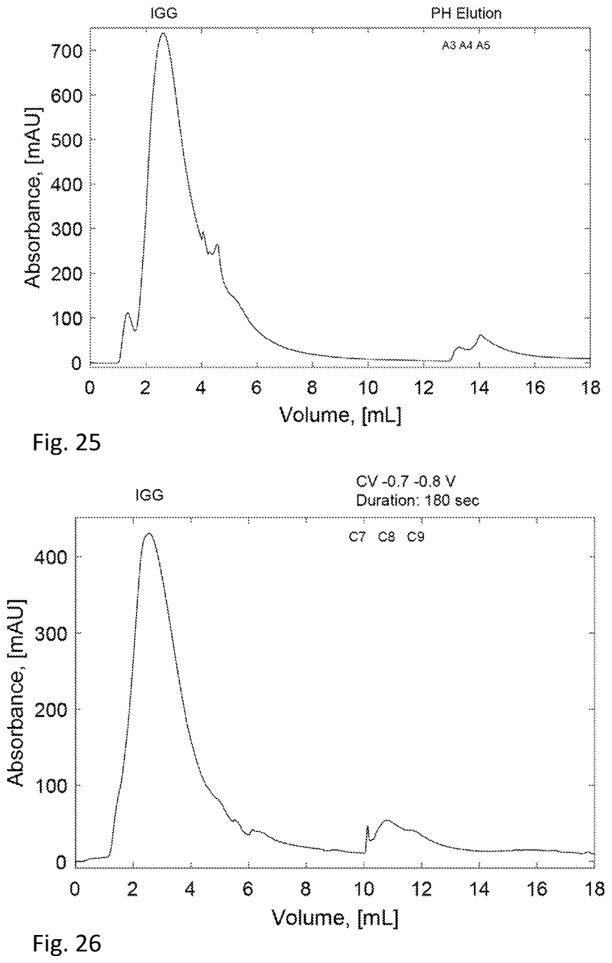

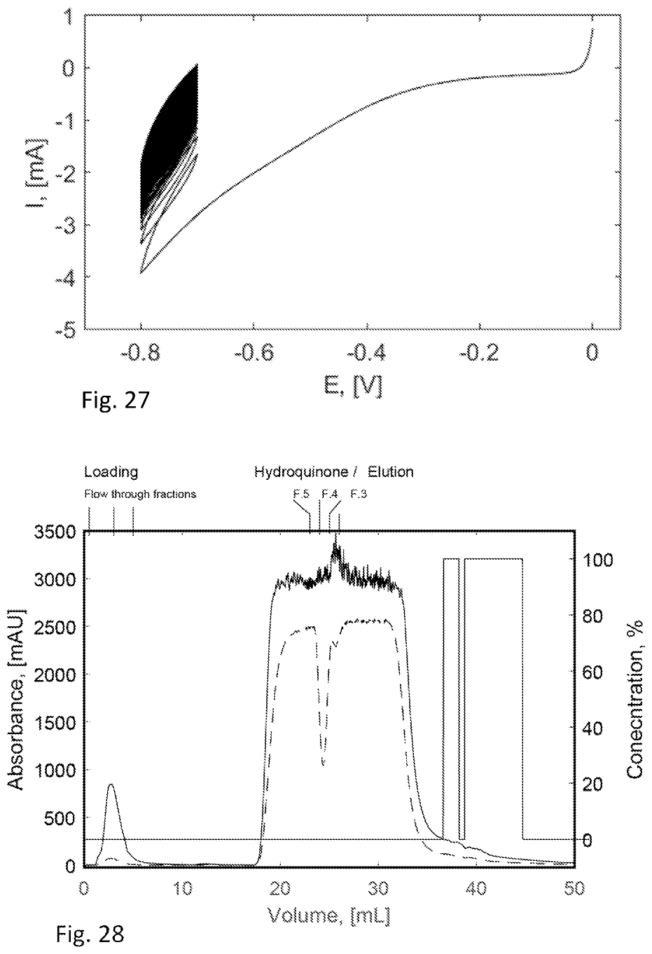

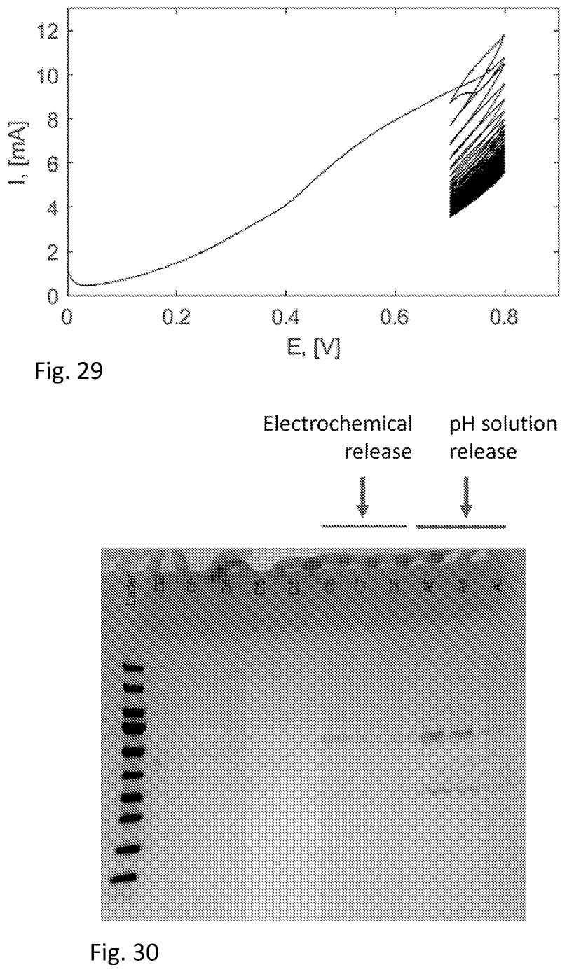

Here we show how microporous electrode supports can be functionalized in an analogous manner as with the QCMD sensors in FIGS. 23 and 24, with Protein A conjugated to a polymer brush coating that enables using the device for electrochemical affinity chromatography. Highly specific interactions with IgG and other monoclonal antibodies were used to enhance the specificity of the capture step followed by non-invasive electrochemical release. First, in FIG. 25 a pure sample of IgG 0.5 mg/mL is injected through the device with a Protein A functionalized microporous stainless-steel electrode. By changing the solution pH value between 2-3 the bound IgG are eluted. In FIG. 26 binding of IgG is repeated, but this time released is triggered by an electrochemical signal. The cyclic voltammetry scan used to achieve elution is shown in FIG. 27, produces a local high pH value on the surface of the electrode. Upon comparison of the elution peaks in FIGS. 25 and 26, the area under the curve of the peak, it is comparable confirming that the quantity of antibodies eluted by electrochemistry is equal to the solution pH eluted amount. Electrochemical signals were also used to produce a local acidic pH gradient within the device as is shown in FIG. 28. Acidic gradients require use of hydroquinone which strongly absorbs UV light used to detect elution in the chromatograms, making it difficult to compare the elution in real time, nevertheless, it was possible to confirm electrochemical activity as the UV signals fluctuate when hydroquinone reacts within the device while the signal was applied. The CV scan used to produce acidic pH gradients is shown in FIG. 29.

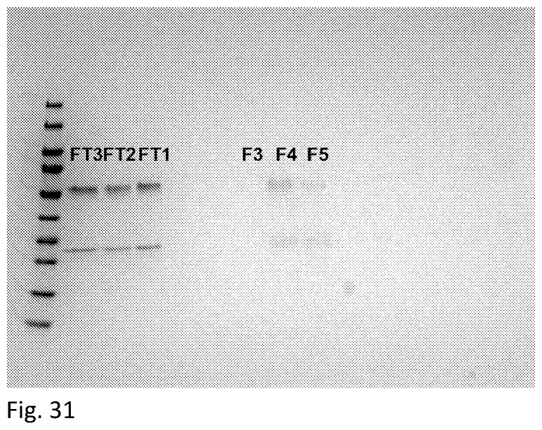

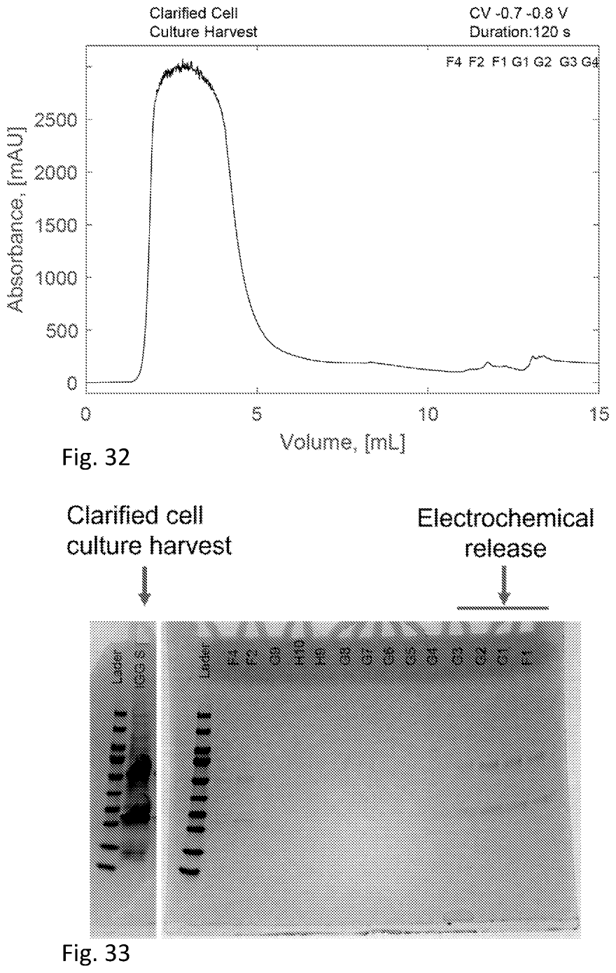

To confirm elution of antibodies sample fractions were collected and SDS-PAGE analysis was performed. FIG. 30 shows bands from antibodies eluted by basic electrochemical signals compared with pH solution elution and in FIG. 31 samples from acidic electrochemical elution is compared to the flow-through through the device of unbound antibodies. The bands for electrochemical elution are fainter than those obtained for pH elution and for flow-through. Optimization of the electrochemical signal may lead to more efficient release of the entire bound quantity of antibodies. The experiment shows that electrochemistry can partially or completely replace use of chemicals to achieve elution. In the case of affinity purification of antibodies this means replacing acid buffers which have been linked to denaturation and agglomeration of the product resulting in substantial yield loss.

In another test, FIG. 32, supernatant, clarified cell culture harvest containing IgG and impurities from the bioprocess was used to demonstrate purification. The sample was loaded onto a protein A functionalized polymer brush on a porous metal mesh. Using electrochemistry IgG was purified from the impurities in the supernatant. SDS-PAGE analysis (FIG. 33) shows several bands from IgG present in samples collected during application of electrochemical signals. Confirming that highly specific interaction (99%) can be combined with electrochemical elution without use of solution pH gradients for antibody purification. Protein A is only one of many ligand interactions which have a strong pH dependence. Almost all biological interactions display a pH profile, or can be engineered to have a pH dependence. This paves the way for testing affinity chromatography of other specialized targets with electrochemical elution, in particular for cases where there is a risk that the elution chemical causes degradation and/or agglomeration of the target molecule.

### Example 10: Concentration by Electrochemistry of a Dilute Protein Sample into a Highly Concentrated Sample

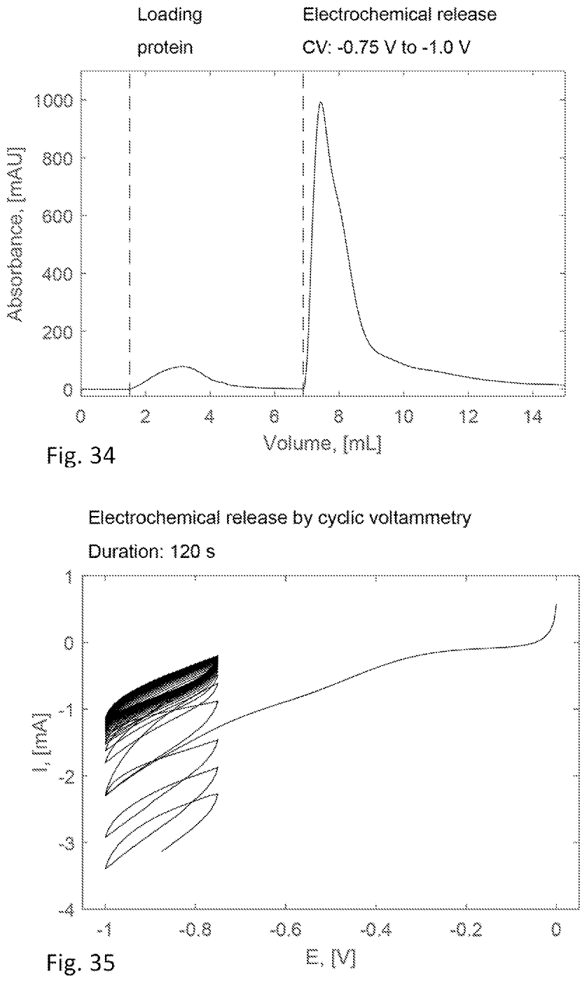

FIG. 34 shows a chromatogram where a dilute protein sample (BSA) is injected through the device where the majority of the proteins are captured to the electrode surface, resulting in a small flow-through peak, followed by a large, sharp peak produced by an electrochemical signal (−1.0 to −0.75 V for 120 s), see FIG. 35 where current is measured against applied voltage. The sample concentration was originally 0.05 g/L and the collected elution peak sample concentration was measured to be 1 g/L, resulting in a 20×-fold increase of the sample concentration. Of the original dilute sample 94% of the sample was eluted by electrochemistry and 6% of the protein did not bind to the electrode indicating a very high sample retention, similar to the sample retention of available commercial concentration centrifuge filters.

Current technical methods for changing concentration and buffer composition uses de-salting columns, or size exclusion columns. However, the resulting concentration is usually low, or requires very long time to complete. Batch concentration or buffer exchange involves centrifugation and spin-columns, or dialysis but these are time-consuming and usually result in yield losses. An in-line concentration of sample with minimal losses of sample reduces yield loss and improves productivity.

### Example 11: Electrochemical Purification of Filled Virus Capsids from Empty Virus Capsids

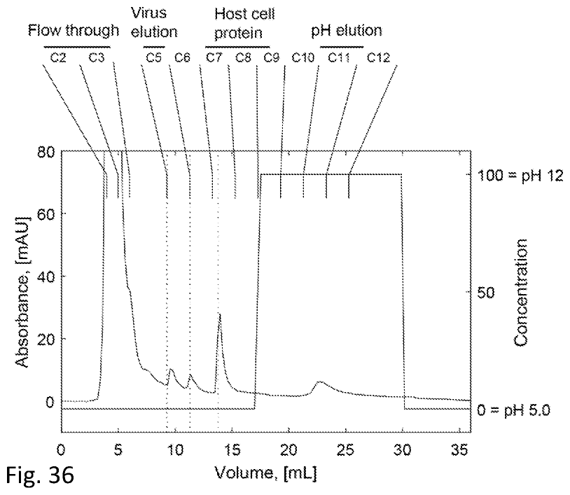

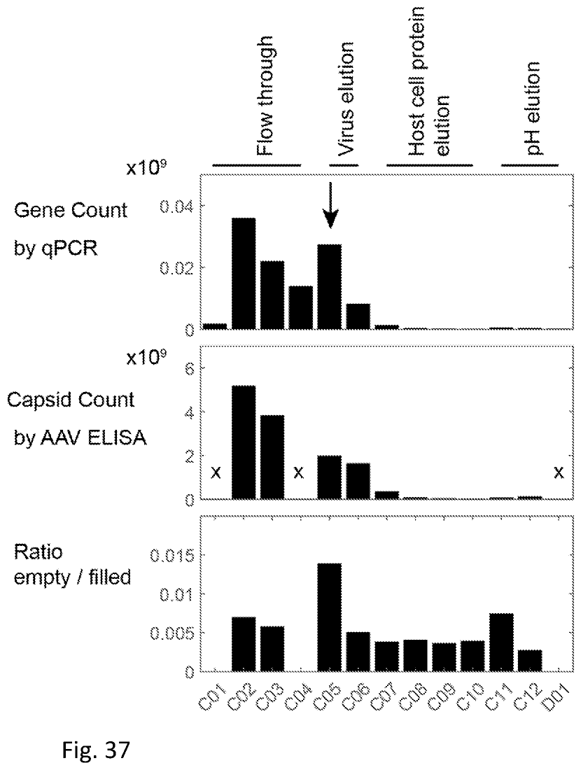

Larger protein constructs than monomer proteins (albumin, IgG, proteins, enzymes) can be purified using electrochemical signals as well. AAVs, non-enveloped virus capsids, were captured on PAA coated microporous stainless-steel electrodes as shown in FIG. 36. Subsequent release by electrochemistry was performed by applying a weak variable electrochemical signal (0 V to −0.5 V) for a duration of 120 s, followed by another signal with the same settings, followed by a stronger electrochemical signal was applied with a larger potential window (0 V to −0.75 V) for a duration of 120 s. Finally, a solution pH step was injected to remove any remaining capsids from the electrode surface. During the application of electrochemical signals samples were collected and analysed for protein capsid content ELISA and a qPCR test, results are shown in FIG. 37, to determine the amount of filled and empty protein capsids in each of the collected samples. The chromatogram in FIG. 36 shows that the largest quantity of biomolecules were collected for in the final, stronger electrochemical signal. However, the smallest quantity of virus capsids was detected in the samples collected for the strong electrochemical signal and the pH solution elution. The probable explanation is that the final elution peak is primarily composed of host-cell-proteins and not virus capsids. Notably the most virus capsids were detected in the samples collected during for the application of two weak signals confirming that the virus capsids were separated from the host-cell protein. Furthermore, a higher concentration of filled virus capsids was detected in the first electrochemical signal, the ratio of filled over empty capsids exceeded the supernatant value demonstrating concentration of filled capsids.

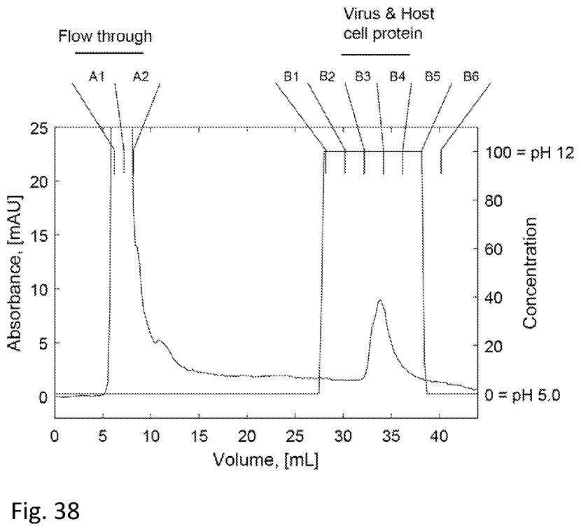

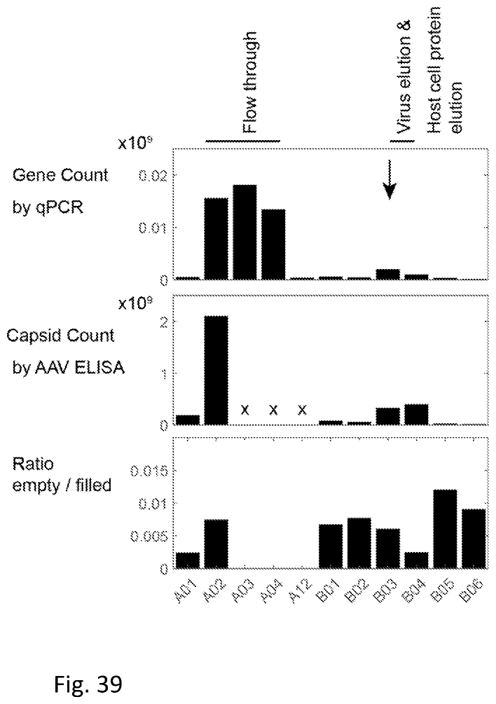

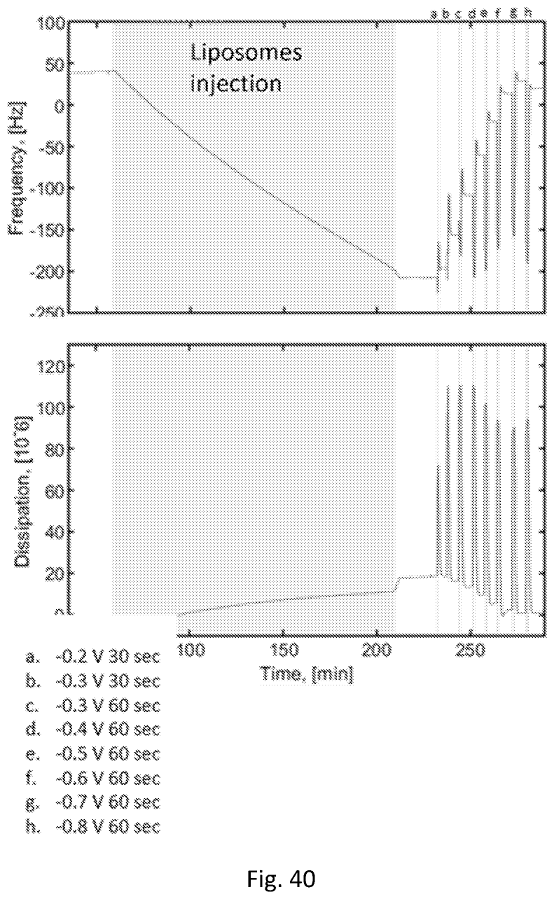

FIG. 38 shows a chromatogram where solution pH was used to trigger release was performed as comparison. In this experiment a single peak is produced, containing host-cell proteins, filled and empty virus capsids.

Analysis of the fractions collected during the solution pH elution confirms elution of AAV capsids. The ratio between filled and empty capsids varies between the samples collected from the same elution peak. It indicates some separation between filled and empty capsids occurs. However, there is not a pronounced concentration of filled capsids observed in any of the samples collected, furthermore there is not a clear sign of separation from host-cell-proteins.

Like with capture of proteins FIG. 18-19, we notice that electrochemical signals can be used to separate viral particles from a supernatant based on their physiochemical properties, charge, size, chemical features. The electrochemical separation is in fact sharper compared to conventional solution ion-exchange by pH or salt concentration gradient.

### Example 12: Lipid Nanoparticle Capture and Electrochemically Mediated Release

So-far data showing analytical sensor-scale and preparative-scale chromatography separation of protein-based analytes has been demonstrated. FIG. 32 shows capture and electrochemical release of lipid-based nanoscale objects. Liposomes, with a mean diameter of 120 nm containing lipids with either charged ionized head groups 5% (mol) or PEG-2000 kDa chains, and predominantly neutral lipids 95% (mol) are first loaded onto a poly(methacrylic acid) functionalized QCMD sensor, followed by release when applying a series of potentials with different magnitudes until the sensor signal return to the baseline indicating complete release. Released Liposomes and liposomes that never bound to the surface were collected and analysed. In Table 3 the diameter of the stock liposomes are compared to the diameter of released liposomes. The released liposomes were slightly larger in diameter, but essentially unharmed from binding and unbinding to the polymer brush surface validating that the method is gentle and non-invasive to large biologically relevant samples which are held together by non-covalent interactions.

TABLE 3

| Potentional (V) | Average Size(nm) | Concentration (particles/ml) |
|---|---|---|
| 0.2-0.3 | 187.9 (+/−4) | 1.18 10⁹ (+/−2.95 10⁷) |
| 0.4-0.5 | 165.6 (+/−4.7) | 1.61 10⁹ (+/−1.59 10⁸) |
| 0.6-0.7 | 180.7 (+/−2.6) | 5.37 10⁸ (+/−7.13 10⁷) |
| 0.8 | 196.2 (+/−3.3) | 4.51 10⁸ (+/−3.10 10⁷) |
| Stock solution | 124.5 (+/−0.3) | 3.53 10⁸ (+/−5.58 10⁷) |

### Example 13: Replacing the Anionic PAA Coating with a Cationic Coating e.g. PDEA (Poly(2-Dimethylamino Methyl) Methacrylate) for Electrostatic Capture and Electrochemical Release of Oligonucleotides or Carbohydrates

Proteins spontaneously bind to PAA in the neutral state and desorb upon triggering charging of the carboxylic acids, or other ligands attached by post-functionalization, by application of electrochemical signals. Oligonucleotides like mRNA and single and double stranded DNA are permanently negatively charged molecules that do not spontaneously bind to neutral PAA and is repelled by a negatively charged polymer coating. This means that there is no state when PAA spontaneously binds oligonucleotides. However, by preparing a cationic polyelectrolytic coating like PDEA oligonucleotides may be spontaneously bound to positively charged tertiary amine functional groups, and with electrochemical signals the coating can be switched into a neutral state suppressing the electrochemical attraction between the oligonucleotide and the coating resulting in electrochemically mediated release. Oligonucleotides are similar to proteins a commonly used biotherapeutic where chromatography is the conventional purification method. The device described in this work may with little modification be used to purify DNA, RNA and different oligonucleotide derivatives as well as therapeutically relevant carbohydrates and glycans like heparin, hyaluronic acid, glycosaminoglycans, and dendritic glycerol sulphate. In principle the device and coating described here may be adapted to purify any large and/or charged macromolecule of interest using electrochemistry where knowledge of the target analyte charge profile as function of pH is used to find at least one binding state and one release state.

### DISCUSSION

Conventional chromatography is used in the production process for all types of biopharmaceuticals, monoclonal antibodies, protein-glycan (carbohydrate) conjugates, oligonucleotide to protein conjugates, bispecific antibodies, enzymes, exosomes, carbohydrates, viral particles, RNA and DNA and even cells, and it is used at all scales from analytical mL scale to industrial 1000s L scale. However chromatography comes with several limitations contributing to high production costs, large water, chemicals and consumables use and leads to long production times.

The device for separation of biological molecules described above incorporates a novel purification mechanism that separates and concentrates the analyte simultaneously in a non-invasive way. The device is in-line connectable with current commercial systems and instruments for separation. The device has a non-trivial design that optimizes capture and release of biomolecules from a polyelectrolytic coating from microporous electrodes using electrochemistry, while in combination permitting liquid flow-through with highly efficient mass-transport of the analyte with low-dilution. It is demonstrated to function on large scale (from mg to g scale) using abundant materials making industrial preparative production with this technology feasible. The scope for analytes that can be separated by the device is uniquely broad spanning protein and/or lipid-containing analytes, oligonucleotides and carbohydrates, and with large variation in sizes of the analyte, which can be from ˜1 nm, or up to several hundred of nm in diameter.

Separation by electrochemical signals offer distinct advantages compared to the traditional separation method of biomolecules by chromatography. The primary advantage is that the chemical change required for tuning binding and release of biomolecules is limited to the chemical microenvironment on the electrode surface, providing a very rapid elution mechanism, with temporary exposure to conditions that trigger elution. In contrast, current methods of chromatography require a time-consuming elution step by flowing a different buffer through the entire column to alter the interaction between the solid support and the biomolecule.

Furthermore, ion-exchange and hydrophobic chromatography requires very high salt concentrations or the need of adding surfactants that later needs to be removed by buffer exchange and dialysis. In the case of affinity chromatography chemicals additives are used which adds a risk of undesired side-reactions with the biomolecule and even denaturation resulting in reduced yield. The present device shows that it would be possible to replace invasive elution protocols used in affinity chromatography with electrochemical elution. For instance, removal of imidazole as elution agent in His-tag purification of recombinant proteins, or removal of extremely acidic pH solution (pH 2-3) washes during antibody separation when performing Protein A chromatography. Electrochemical elution removes the need of post-processing to remove unwanted components in the product feed like, acidic pH, salt, surfactants and chemicals like imidazole.

In some cases, the current methods of chromatography fail to provide acceptable yield and purity, due to harsh and invasive purification methods, prolonging the time to market for promising new biopharmaceuticals. The device described above, which utilizes electrochemical elution offers a very brief treatment without any chemical additives serving as an alternative method for production of challenging target analytes.

Preparative purification by electrochemistry places two primary conditions on the design of the device. First is optimization with respect to electrochemistry. We have found that for optimization of electrochemistry it is important to have an interior volume of the device is large enough that the electrodes are physically separated and with a void gap space permitting liquid to flow. Furthermore, the counter electrode needs to be capable of sustaining the specified voltage on the working electrode. Secondly the overall interior volume of the device needs to be small enough that the liquid volume does not dilute the product more than necessary. A trade-off between the electrochemical properties of the multi-electrode cell, and the contribution of dilution by excessive void volume needs to be found. Splitting the electrode cell into two compartments, one for the working electrode, and one for the counter electrode, removes of at least half of the from the liquid volume permitting substantial improvement of the product concentration. However, even for designs where the electrodes are placed into a single compartment productivity comparable or exceeding chromatography products could be obtained. For example, in-line concentration of samples with 94% sample retention was shown to be possible which would limit the need to perform buffer exchange and up-concentration. Concentration and buffer exchange are two off-line operations commonly used in bioproduction where concentration is done by centrifugation, and buffer exchange by dialysis. Off-line process steps increase the production time and cause large yield losses.

Binding capacity of the material that captures biomolecules is a key performance metric for preparative purifications. The device described herein comprises a working electrode, which is coated with a stimuli-responsive polyelectrolyte coating, such as a polyelectrolytic brush. The polyelectrolytic coating offers the advantage of binding large quantities of analytes per surface area. Hydrophilic polyelectrolytic coatings (e.g. polyacrylic acid) produce a soft three-dimensional scaffold that preserve protein structure and with exceptional surface coverage capacity in the range of ˜μg/cm². In contrast, chromatography resins, e.g. agarose or acrylamide, are surface activated to directly bind protein in monolayers on the resin surface.

The binding capacity of chromatography solid supports are boosted by adjusting the pore and particle size of the resin beads resulting in larger surface area. However, the surface area and porosity can be tuned for any solid support, also for those functionalized with polymer brushes. Extremely small porosity eventually introduces other problems such as mass-transfer and flow limitations and difficulties in cleaning. By using the polyelectrolytic coating to gain high surface binding capacity, the device described herein can offer better mass-transport properties than traditional devices. Furthermore, the shape of the electrode can be adjusted to give the device desirable features.

Contrary to the random internal structure of chromatography materials and the random packing of particles within the chromatography column, the electrodes materials used in this device, provide a more ordered structure that may contribute to a more predictable and even flow pattern through the device compared to a chromatography column. Many conductive materials can be considered for working electrodes since the aryl bond produced by diazonium salt deposition is versatile and works on steel, carbon, gold, platinum, aluminium, silicon and other semiconductors.

Use of stimuli-responsive polyelectrolyte coating enables multiple modes of interaction with the analyte. A stimuli-responsive polyelectrolyte can interact with a biological analyte by non-electrostatic attraction, electrostatic attraction, and electrostatic repulsion. As exemplified, the polyelectrolytic coating of the device displays these modes of interactions with analytes where attraction and repulsion is controlled, rapid, non-invasive electrochemical signals. These signals separate analytes by multiple different kinds of molecular properties of the analyte (so-called multimodal separation) through a combination of electrostatic interactions (ion-exchange) and mild hydrophobic interactions (hydrogen bonding). By changing the properties of the solution, changing the pH and the salt concentration primarily, allows for changing the conditions for the interaction. By engineering of the chemical identity of the polyelectrolyte coating it is highly probable that the device can be adjusted to capture and release different types of analytes. Other polyelectrolytic coatings than PAA brushes can be used, such as: poly(carboxybetaine methacrylamide) (PCBMAM), poly(2-diethylamino)ethyl methacrylate) (PDEA), monomers with amino acid side groups e.g, poly(serine methacrylate) (PSMA), The chemical identity of the polyelectrolytic coating can be allowed to vary further as the coating may be post-functionalized to carry biological ligands e.g. peptides, affinity tags, protein A/G, calmodulin, by using bioconjugation techniques like EDC/NHS of carboxylic acid and amine functional groups of the polyelectrolytic coating. Furthermore, polyelectrolytic coatings with highly specific biological ligands can be prepared from initially neutral coatings like: PGMA and PHEMA by binding biological ligands to functional groups like epoxy groups. Biopolymers could also be used as coatings where examples of such polymers are: hyaluronic acid, heparin, dextran. The polyelectrolytic coating may be a polymer brush, but also other coatings than brushes could be used where a dense polymer coating is produced, such as a gel, for example a hydrogel, a cross-linked layer by layer coating. The main requirement in the development of the working electrode is to ensure that the polymer coating is sufficiently strongly bound (here by covalent bonds) to the working electrode surface, such that it is able to tolerate the electrochemical signals that trigger binding affinity changes between the analyte and the solid support.

A combination of the physical and chemical properties of the polyelectrolytic coating can be adjusted to meet the requirements for a specific biopurification process. For instance, carbohydrates, oligonucleotides are larger less compact molecules compared to proteins. By preparation of a polymer brush that has sparser grafting density, the polyelectrolytic coating can permit highly efficient binding of larger molecules to the surface in multi-layers. In combination, the polyelectrolytic coating chemistry can be adjusted to fit a situation where spontaneous binding of the analyte is achieved, either by electrostatic attraction or by non-covalent interactions, and where release is achieved for the opposite state.

The grafting density of the polyelectrolytic coating can be an important factor for promoting binding of large molecules to ensure enough void space within the polyelectrolytic coating for efficient intercalation within the polymer layer in multilayers. Sufficient porosity of the underlying scaffold is also important. For instance, in production of viral vectors from gene therapy is challenging due to lack of good options for purification contributing to production costs. Chromatography materials optimized for monomeric proteins have too fine pores that risks clogging of the resin, and use of elution chemicals is complicated as the non-covalent adhesion between the capsid proteins that make up the virus construct is easily disrupted by elution chemicals such as pH, salt and surfactants. In gene therapy carriers typically have a very low fraction of particles loaded with genetic material. Empty capsids and empty carrier nanoparticles are a patient security risk, increasing the risk of serious allergic reactions and immunogenic responses and necessitate the use of highly concentrated injections with low efficacy. There are currently no good tools for affinity chromatography (highly specific purification methods) that can separate filled from empty carrier nanoparticles.

Some viral vectors considered for gene therapy, for instance lentiviruses are enveloped, meaning they have a lipid bilayer outer shell. Biological targets such as enveloped viral vectors and exosomes are becoming increasingly relevant as carrier materials for gene therapy. These biological constructs are predominantly built with lipids. In addition to purification of protein-based analytes we also demonstrate capture and release by electrochemical signals of lipid-based targets.

Finally, the device offers substantial improvement of a currently very inefficient production process. The device herein has the potential to substantially reduce use of water, time and chemicals which would reduce cost of goods for production, accelerate the speed of production and reduce the climate impact of biopharmaceutical production. A consequence of using the device could be the faster development time of new biotherapeutics, higher accessibility of biopharmaceuticals.

1. A device (100, 100′, 100″) for separating an analyte (200) from other components in an electrolytic solution, the device comprising:
a housing (114, 115, 116, 117, 118, 119) provided with a solution inlet (104) and a solution outlet (105),
a working electrode (101) arranged in the housing (114, 115, 116, 117, 118, 119) in a space between the solution inlet (104) and the solution outlet (105), and arranged such that an electrolytic solution arranged to flow (F) from the inlet to the outlet contacts at least a portion of the working electrode,
a counter electrode (102) arranged in the housing (114, 115, 116, 117, 118, 119) in a space between the inlet (104) and the outlet (105) at a distance from the working electrode (101), and arranged such that it is in electrical connection with the working electrode via the electrolytic solution arranged to flow from the inlet to the outlet,
wherein at least a portion of a surface of the working electrode (101) is provided with a polyelectrolytic coating (111), the polyelectrolytic coating (111) being arranged to upon application of a potential difference between the working electrode (101) and the counter electrode (102) switch between a first and second state, wherein in the first state an analyte (200) is captured in said polyelectrolytic coating (111) and in the second state a captured analyte (200) is released from said polyelectrolytic coating (111).

2. The device (100, 100′, 100″) of claim 1, wherein the analyte is selected from a protein, a lipid particle, an oligonucleotide, a carbohydrate, or any combination thereof.

3. The device (100, 100′, 100″) of claim 1, wherein the polyelectrolytic coating arranged on the surface of the working electrode comprises a pH-responsive polymer covalently bound to the surface of the electrode through a monolayer of aryl bonds.

4. (canceled)

5. The device (100, 100′, 100″) of claim 3, wherein the pH-responsive polymer is a polymer functionalized with a pH-responsive and analytespecific ligand.

6. (canceled)

7. The device (100, 100′, 100″) of claim 1 wherein an average distance between the working electrode (101) and the counter electrode (102) ranges from 20 pm to 20 mm.

8. The device (100, 100′, 100″) of claim 1, wherein an average thickness of the polyelectrolytic coating provided on the working electrode (101) is 10-50 nm.

9. The device (100, 100′, 100″) of claim 1, wherein 70-100% of the working electrode (101) overlaps with the counter electrode, as seen in a plane orthogonal to a direction of flow (F) of the electrolytic solution from the solution inlet (104) towards the solution outlet (105).

10. The device (100, 100′, 100″) of claim 1, wherein the inner volume of the housing (114, 115, 116, 117, 118, 119) not occupied by the working electrode (101) is 5%-75%.

11. The device (100, 100′, 100″) of claim 1, wherein the working electrode (101) is porous and arranged in the housing (114, 115, 116, 117, 118, 119) such that the electrolytic solution is allowed to flow from the inlet (104) through at least a portion of the working electrode (101) to the outlet (105) and wherein the working electrode has a porosity of 40% to 99%, and an electroactive surface area of the working electrode is between 100 to 10,000 m²/m³.

12. (canceled)

13. The device (100, 100′, 100″) of claim 11, wherein the counter electrode (101) is porous.

14. The device (100, 100′, 100″) of claim 11, wherein the working electrode (101) and the counter electrode (102) are arranged in the housing (114, 115, 116, 117, 118, 119) such that the electrolytic solution arranged to flow (F) from the inlet to the outlet first passes through the working electrode (101) and then through or past the counter electrode (102).

15. The device (100, 100′, 100″) of claim 11, wherein a void space within the working electrode (101) is configured such that electrolytic solution passing through the working electrode (101) creates an electrochemical pH gradient that is at least 1-20 pm large.

16. (canceled)

17. The device (100, 100′, 100″) of claim 1, further comprising a reference electrode arranged in the housing and arranged for electrical connection through the electrolyte solution with the working electrode and the counter electrode, wherein the reference electrode (103) is arranged at an average distance of 1-50 mm from the counter electrode (102) and at an average distance of 1-50 mm from the working electrode (101).

18. The device (100, 100′, 100″) of claim 1, further comprising an ion-selective membrane (106) arranged between the working electrode (101) and the counter electrode (102) in the housing (114, 115, 116, 117, 118, 119).

19. (canceled)

20. The device (100, 100′, 100″) of claim 1, wherein an effective surface area of the counter electrode (102) is at least two times larger than an effective surface area of the working electrode (101).

21. The device of claim 1, comprising two connected chambers (117, 118), one chamber (118) for the working electrode (101) and one chamber (117) for the counter electrode (102), separated by an ion-permeable membrane.

22. A system (300) for separating an analyte (200) from other components in an electrolytic solution, the system comprising:
the device (100, 100′, 100″) of claim 1, and
an arrangement (301) for applying a potential difference between the working electrode (101) and the counter electrode (102),
a flow system arranged to supply the electrolytic solution to the housing (114, 115, 116, 117, 118, 119) at the solution inlet (104),
a solution collection system (302) arranged at the solution outlet (105) of the housing (114, 115, 116, 117, 118, 119) for collecting solution and analyte exiting the device (100, 100′, 100″) through the solution outlet (105).

23. (canceled)

24. A method of separating an analyte (200) from other components in an electrolytic solution, comprising:
providing a system (300) of claim 22,
providing an electrolytic solution comprising an analyte (200) to be separated from other components in the electrolytic solution,
supplying the electrolytic solution comprising the analyte (200) to the housing (114, 115, 116, 117, 118, 119) at the solution inlet (104),
allowing the solution to flow from the inlet (104) to the outlet (105) such that the analyte is captured by the polyelectrolytic coating (111) arranged on the working electrode (101),
applying a potential difference between the working electrode (101) and the counter electrode (102), thereby releasing the analyte (200) from said polyelectrolytic coating (111) and eluting the analyte (200) from the working electrode (101), collecting solution comprising the analyte (101) exiting through the solution outlet (105).

25. The method of claim 24, comprising a step before supplying the electrolytic solution comprising the analyte (200) to the device (100, 100′, 100″) of running a buffer through the device (100, 100′, 100″), the running buffer having a pH between pH 4 to pH 8.

26. (canceled)

27. The method of claim 24, wherein when applying a potential difference between the working electrode (101) and the counter electrode (103) for releasing the analyte (200) from the polyelectrolytic coating (111) and eluting the analyte (200) from the working electrode (101), a running buffer flow rate of 0 mL/min to 10 mL/min is used.

28.-33. (canceled)
